(19) United States
(12) Patent Application Publication
(10) Pub. No.: US 2018/0238908 A1
(43) Pub. Date: Aug. 23, 2018

(54) NEURODEGENERATIVE DISORDERS

(71) Applicant: KRAN LIFE SCIENCES LLP, Epsom (GB)

(72) Inventor: Krtin Kanna NITHIYANANDAM, Epsom (GB)

(21) Appl. No.: 15/750,327

(22) PCT Filed: Jul. 21, 2016
(86) PCT No.: PCT/GB2016/052206

§ 371 Date: Feb. 5, 2018

(30) Foreign Application Priority Data
Aug. 5, 2015 (GB) 1513845.6

(51) Int. Cl. G01N 33/68; A61K 47/68; A61K 47/69; A61K 49/00; A61K 49/04; A61K 49/10; A61K 49/18

(52) U.S. Cl. CPC G01N 33/6896; A61K 47/6879; A61K 47/6923; A61K 47/6929; G01N 2333/4709; A61K 49/0428; A61K 49/108; A61K 49/1824; A61K 49/0002

(57) ABSTRACT

An amyloidogenic peptide biospecific agent comprises a nanoparticle which is visible under near infrared (NIR) and/or using Magnetic Resonance Imaging (MRI) and/or Computed Tomography (CT). The biospecific agent further comprises at least one antibody or antigen binding fragment thereof, which is immunospecific for a transferrin receptor and an amyloidogenic peptide.

The invention relates to neurodegenerative disorders, and in particular to novel compositions, therapies and methods for diagnosing and treating such conditions, including Alzheimer's disease, Parkinson's disease and Huntington's disease.

The term “neurodegenerative” is broadly used for the progressive loss of structure and/or function of neurons. Many neurodegenerative diseases, including Alzheimer's, Parkinson's and Huntington's disease, occur as a result of neurodegenerative processes, and are currently incurable, resulting in progressive degeneration and/or death of neurons. A common feature which links many neurodegenerative diseases is that they all involve the accumulation of amyloids, which are fibrous protein aggregates sharing specific structural traits. Amyloids are insoluble and arise from at least 18 inappropriately folded versions of proteins and polypeptides present naturally in the body. These misfolded structures alter their proper configuration such that they erroneously interact with one another or other cell components forming insoluble fibrils. To date, amyloids have been associated with the pathology of more than 20 serious human diseases in that abnormal accumulation of amyloid fibrils in organs may lead to amyloidosis.

Alzheimer's disease, for example, is characterized by the deposition of Amyloid Beta (Aβ) in extracellular amyloid plaques, as well as the intracellular accumulation of tau in neurofibrillary tangles in the brain. Mutations in Aβ and the Amyloid Precursor Protein (APP) are linked to familial Alzheimer's disease, and therefore Aβ is thought to play an important role in the disease process. Certain members of the Aβ family are toxic, most notably Aβ oligomers, and have been shown to cause membrane defects, neuronal cell death and effects on function, and to lead to changes in animal behaviour and neuronal networks. The Aβ peptide is a member of a larger group of amyloidogenic peptides and proteins, and it is believed that the toxic effect of these amyloidogenic peptides is linked to their ability to self-assemble to form β-sheet rich oligomeric species and cross-β structured amyloid fibrils. The amyloid-based peptides responsible for Parkinson's disease and Huntington disease are alpha-synuclein and Huntingtin, respectively.

Currently available diagnostic tools are able to detect these amyloid proteins, including Aβ plaques, synuclein and Huntingtin etc., which are present during the later stages of the corresponding disease, and so are unable to focus on early-stage identification of the conditions. Therefore, there is a significant need to provide novel means for the early diagnosis and/or treatment neurodegenerative diseases, such as Alzheimer's, Parkinson's and Huntington's disease.

The inventor has now developed a novel neurodegenerative disorder diagnostic and therapeutic tool, which exhibits high specificity for (i) the transferrin receptor, such it can be readily transported across the blood brain barrier via receptor-mediated transcytosis, and (ii) a range of different amyloidogenic peptides, such as Aβ, synuclein and Huntingtin.

Thus, according to a first aspect of the invention, there is provided an amyloidogenic peptide biospecific agent comprising a nanoparticle which is visible under near infrared (NIR) and/or using Magnetic Resonance Imaging (MRI) and/or Computed Tomography (CT), and at least one antibody or antigen binding fragment thereof, which is immunospecific for a transferrin receptor and an amyloidogenic peptide.

As described in the examples, the biospecific agent of the invention displayed a very low cytotoxicity whilst exhibiting the ability to target intracellular amyloid-beta (Aβ) species. The biospecific agent displayed low cross-reactivity with Aβ monomers and plaques, whilst displaying a high affinity to amyloid-beta oligomers and fibrils, such that it can be used to achieve much earlier diagnosis of neurodegenerative disorders than is possible using currently available diagnostic tools. The biospecific agent also displayed a low affinity to transferrin receptors, once targeted thereto by the bispecific antibody, which is required for efficiently crossing the blood brain barrier via receptor-mediated transcytosis. The biospecific agent of the invention emits light in the near infrared (NIR), maximally at about 850 nm, due to its chemical composition, such that it can be visualised at tissue depths of around 2 cm, which is ideal for in vivo diagnosis of subjects suffering from, or thought to suffer from, a neurodegernative disorder. Furthermore, the composition of the agent, as described in the examples, significantly improves the biocompatibility of the agent and drastically decreased its toxicity, and allows for detection via MRI or CT. In addition to its surprisingly robust diagnostic ability, the inventor was also surprised to observe that the agent displayed therapeutic effects as the agent was able to bind oligomers of the amyloidogenic peptide, which were therefore less readily able to enter neurons due to them being rendered insoluble, as displayed by the immunofluorescence.

Due to the presence of the antibody or antigen binding fragment thereof which targets and specifically binds to the amyloidogenic peptide, the biospecific agent of the invention can be used to target any amyloidogenic peptide or protein acting as a biomarker for any neurodegenerative disease, for example Aβ in Alzheimer's disease, alpha-synuclein in Parkinson's, or Huntingtin in Huntington's disease. The agent is highly suitable for the sensitive and non-invasive detection and imaging of amyloidogenic peptides, and therefore early diagnosis of neurodegenerative diseases. Early detection of such diseases means that treatment regimes can be initiated much sooner than is possible without currently available techniques, and before symptoms start to show by focusing on pathophysiological changes, some of which can occur a decade before symptoms are prevalent. This early diagnosis can help patients and their families prepare for the future, allowing them to choose to enter clinical trials for potentially life-saving drugs at an earlier stage, and ensure that existing drugs are used to better effect, such that patients have a better prognosis.

Preferably, the at least one antibody or antigen binding fragment comprises an IgG anti-amyloidogenic peptide antibody or antigen binding fragment thereof. Preferably, the moiety of the bispecific agent which is immunospecific for an amyloidogenic peptide comprises an antibody fragment, more preferably a Fab′ fragment.

Preferably, the at least one antibody or antigen binding fragment binds specifically to oligomers and fibrils (including protofibrils) of the amylodogenic peptide, but not to amyloidogenic peptide plaques and peptide monomers. Advantageously, the antibody's specificity for amyloidogenic peptide oligomers and fibrils, but not amyloidogenic peptide plaques and monomers, means that the biospecific agent of the invention can be used to detect early stage neurodegenerative disease.

For example, in one embodiment, the biospecific agent of the invention may be used to detect and treat Alzheimer's disease. Preferably, therefore, the immunogen sequence used to create the at least one antibody or antigen binding fragment against the amyloidogenic peptide comprises or consists of the amino acid sequence of Aβ, or a variant or fragment thereof. The amino acid sequence of wild-type Aβ(1-42) is known, and may be represented herein as SEQ ID No:1, as follows:—

[SEQ ID No: 1]
DAEFRHDSGYEVHHQKLVFFAEDVGSNKGAIIGLMVGGVVIA

Preferably, the immunogen sequence used to create the at least one antibody or antigen binding fragment against the amyloidogenic peptide comprises or consists of SEQ ID No:1, or a variant or fragment thereof. The inventor has realised that Aβ oligomers are present in much higher concentrations in the brains of Alzheimer's patients, and this increase appears during the earliest stages of the diseases, making Aβ oligomers a more attractive biomarker than Aβ plaques or monomers of Aβ. Accordingly, preferably the immunogen sequence used to create the at least one antibody or antigen binding fragment against the amyloidogenic peptide comprises or consists of partially aggregated wild-type Aβ(1-42) peptide, more preferably partially aggregated SEQ ID No:1. Preferably, the at least one antibody or antigen binding fragment binds specifically to amyloid beta oligomers and fibrils (including protofibrils), but not to Aβ plaques or monomers. As such, the antibody's specificity for Aβ oligomers and fibrils, but not Aβ plaques and monomers means that the biospecific agent of the invention can be used to detect early stage Alzheimer's disease.

In another embodiment, the biospecific agent of the invention may be used to detect and treat Huntington's disease. Preferably, the immunogen sequence used to create the at least one antibody or antigen binding fragment against the amyloidogenic peptide comprises or consists of the amino acid sequence of Huntingtin, or a variant or fragment thereof. The amino acid sequence of an embodiment of human Huntingtin is known, and may be represented herein as SEQ ID No:2, as follows:—

[SEQ ID No: 2]
matleklmka feslksfqqq qqqqqqqqqq qqqqqqqqqq
pppppppppp pqlpqpppqa qpllpqpqpp ppppppppgp
avaeeplhrp kkelsatkkd rvnhcltice nivaqsvrns
pefqkllgia melfllcsdd aesdvrmvad eclnkvikal
mdsnlprlql elykeikkng aprslraalw rfaelahlvr
pqkcrpylvn llpcltrtsk rpeesvqetl aaavpkimas
fgnfandnei kvllkafian lksssptirr taagsavsic
qhsrrtqyfy swllnvllgl lvpvedehst llilgvlltl
rylvpllqqq vkdtslkgsf gvtrkemevs psaeqlvqvy
eltlhhtqhq dhnvvtgale llqqlfrtpp pellqtltav
ggigqltaak eesggrsrsg siveliaggg sscspvlsrk
qkgkvllgee ealeddsesr sdvsssalta svkdeisgel
aassgvstpg saghdiiteq prsqhtlqad svdlascdlt
ssatdgdeed ilshsssqvs avpsdpamdl ndgtqasspi
sdssqttteg pdsavtpsds seivldgtdn qylglqigqp
qdedeeatgi lpdeaseafr nssmalqqah llknmshcrq
psdssvdkfv lrdeatepgd qenkperikg digqstddds
aplvhcvrll sasflltggk nvlvpdrdvr vsvkalalsc
vgaavalhpe sffsklykvp ldtteypeeq yvsdilnyid
hgdpqvrgat ailcgtlics ilsrsrfhvg dwmgtirtlt
gntfsladci pllrktlkde ssvtcklact avrncvmslc
sssyselglq liidvltlrn ssywlvrtel letlaeidfr
lvsfleakae nlhrgahhyt gllklqervl nnvvihllgd
edprvrhvaa aslirlvpkl fykcdqgqad pvvavardqs
svylkllmhe tqppshfsvs titriyrgyn llpsitdvtm
ennlsrviaa vshelitstt raltfgccea lcllstafpv
ciwslgwhcg vpplsasdes rksctvgmat miltllssaw
fpldlsahqd alilagnlla asapkslrss waseeeanpa
atkqeevwpa lgdralvpmv eqlfshllkv inicahvldd
vapgpaikaa lpsltnppsl spirrkgkek epgeqasvpl
spkkgseasa asrqsdtsgp vttskssslg sfyhlpsylk
lhdvlkatha nykvtldlqn stekfggflr saldvlsqil
elatlqdigk cveeilgylk scfsrepmma tvcvqqllkt
lfgtnlasqf dglssnpsks qgraqrlgss svrpglyhyc
fmapythftq aladaslrnm vqaeqendts gwfdvlqkvs
tqlktnltsv tknradknai hnhirlfepl vikalkqytt
ttcvqlqkqv ldllaqlvql rvnyclldsd qvfigfvlkq
feyievgqfr eseaiipnif fflvllsyer yhskqiigip
kiiqlcdgim asgrkavtha ipalqpivhd lfvlrgtnka
dagkeletqk evvvsmllrl iqyhqvlemf ilvlqqchke
nedkwkrlsr qiadiilpml akqqmhidsh ealgvlntlf
eilapsslrp vdmllrsmfv tpntmasvst vqlwisgila
ilrvlisqst edivlsriqe lsfspylisc tvinrlrdgd
ststleehse gkqiknlpee tfsrfllqlv gilledivtk
qlkvemseqq htfycqelgt llmclihifk sgmfrritaa
atrlfrsdgc ggsfytldsl nlrarsmitt hpalvllwcq
illlvnhtdy rwwaevqqtp krhslsstkl lspqmsgeee
dsdlaaklgm cnreivrrga lilfcdyvcq nlhdsehltw
livnhiqdli slsheppvqd fisavhrnsa asglfiqaiq
srcenlstpt mlkktlqcle gihlsqsgav ltlyvdrllc
tpfrvlarmv dilacrrvem llaanlqssm aqlpmeelnr
iqeylqssgl aqrhqrlysl ldrfrlstmq dslspsppvs
shpldgdghv sletvspdkd wyvhlvksqc wtrsdsalle
gaelvnripa edmnafmmns efnlsllapc lslgmseisg
gqksalfeaa revtlarvsg tvqqlpavhh vfqpelpaep
aaywsklndl fgdaalyqsl ptlaralaqy lvvvsklpsh
lhlppekekd ivkfvvatle alswhliheq iplsldlqag
ldccclalql pglwsvvsst efvthacsli ycvhfileav
avqpgeqlls perrtntpka iseeeeevdp ntqnpkyita
acemvaemve slqsvlalgh krnsgvpafl tpllrniiis
larlplvnsy trvpplvwkl gwspkpggdf gtafpeipve
flqekevfke fiyrintlgw tsrtqfeetw atllgvlvtq
plvmeqeesp peedtertqi nvlavqaits lvlsamtvpv
agnpavscle qqprnkplka ldtrfgrkls iirgiveqei
qamvskreni athhlyqawd pvpslspatt galishekll
lqinperelg smsyklgqvs ihsvwlgnsi tplreeewde
eeeeeadapa psspptspvn srkhragvdi hscsqfllel
ysrwilpsss arrtpailis evvrsllvvs dlfternqfe
lmyvtltelr rvhpsedeil aqylvpatck aaavlgmdka
vaepvsrlle stlrsshlps rvgalhgvly vlecdllddt
akqlipvisd yllsnlkgia hcvnihsqqh vlvmcatafy
lienypldvg pefsasiiqm cgvmlsgsee stpsiiyhca
lrglerllls eqlsrldaes lvklsvdrvn vhsphramaa
lglmltcmyt gkekvspgrt sdpnpaapds esvivamerv
svlfdrirkg fpcearvvar ilpqflddff ppqdimnkvi
geflsnqqpy pqfmatvvyk vfqtlhstgq ssmvrdwvml
slsnftqrap vamatwslsc ffvsastspw vaailphvis
rmgkleqvdv nlfclvatdf yrhqieeeld rrafqsvlev
vaapgspyhr lltclrnvhk vttc

Preferably, the immunogen sequence used to create the at least one antibody or antigen binding fragment against the amyloidogenic peptide comprises or consists of SEQ ID No:2, or a variant or fragment thereof. Preferably, the immunogen sequence used to create the at least one antibody or antigen binding fragment against the amyloidogenic peptide comprises or consists of partially aggregated SEQ ID No:2. Most preferably, the at least one antibody or antigen binding fragment binds specifically to Huntingtin oligomers and fibrils (including protofibrils), but not to Huntingtin plaques and monomers. Advantageously, the antibody's specificity for Huntingtin oligomers and fibrils, but not Huntingtin plaques and monomers means that the biospecific agent of the invention can be used to detect early stage Huntington's disease.

In yet another embodiment, the biospecific agent of the invention may be used to detect and treat Parkinson's disease. Preferably, the immunogen sequence used to create the at least one antibody or antigen binding fragment against the amyloidogenic peptide comprises or consists of the amino acid sequence of alpha-synuclein, or a variant or fragment thereof. The amino acid sequence of an embodiment of human alpha-synuclein (e.g. isoform NACP140) is known, and may be represented herein as SEQ ID No:3, as follows:—

[SEQ ID No: 3]
mdvfmkglsk akegvvaaae ktkqgvaeaa gktkegvlyv
gsktkegvvh gvatvaektk eqvtnvggav vtgvtavaqk
tvegagsiaa atgfvkkdql gkneegapqe giledmpvdp
dneayempse egyqdyepea

Preferably, the immunogen sequence used to create the at least one antibody or antigen binding fragment against the amyloidogenic peptide comprises or consists of SEQ ID No:3, or a variant or fragment thereof. Preferably, the immunogen sequence used to create the at least one antibody or antigen binding fragment against the amyloidogenic peptide comprises or consists of partially aggregated SEQ ID No:3. Most preferably, the at least one antibody or antigen binding fragment binds specifically to alpha-synuclein oligomers and fibrils (including protofibrils), but not to alpha-synuclein plaques and monomers. Advantageously, the antibody's specificity for alpha-synuclein oligomers and fibrils but not alpha-synuclein plaques and monomers means that the biospecific agent of the invention can be used to detect early stage Parkinson's disease.

The blood-brain-barrier is a highly selective permeable barrier formed by capillary endothelial cells, and this ensures that very few objects can reach the brain, advantageous for protecting the brain from invading pathogens or toxins, but problematic as very few therapeutic agents are able to pass through to reach the brain. Antibodies with a low affinity to transferrin receptors (TfRs) are able to cross the blood-brain-barrier via receptor-mediated transcytosis. The anti-TfR antibody or fragment thereof targets and binds the nanoparticle to the TfR, and is transported across the endothelial cell, but due to the low affinity of the antibody for the receptor, when it reaches the other side of the endothelial cell, it is released from the TfR and into the brain.

Accordingly, preferably the at least one antibody or antigen binding fragment comprises an IgM anti-transferrin receptor antibody or antigen binding fragment thereof. FIG. 4 shows the results of surface Plasmon resonance for determining the affinity of the biospecific agent of the invention to transferrin receptors. As can be seen, the high Kd value is a clear demonstration of the low affinity to these receptors, important for enabling receptor-mediated transcytosis to occur. Preferably, the binding affinity with the transferrin receptor is in the micromolar range. Preferably, the disassociation constant value of the at least one antibody or antigen binding fragment thereof for the transferrin receptor is at least 1×10⁻⁴M, more preferably at least 1×10⁻³M. Preferably, the affinity constant should be no less than 1×10⁻⁴ M, as affinity constants lower than this may fail to engage the transferrin receptor entirely.

Preferably, the moiety of the bispecific agent which is immunospecific for a transferrin receptor comprises an antibody fragment, more preferably a Fab′ fragment.

The amino acid sequence of one embodiment of a human transferrin receptor is known, and may be represented herein as SEQ ID No:4, as follows:—

[SEQ ID No: 4]
mmdqarsafs nlfggeplsy trfslarqvd gdnshvemkl
avdeeenadn ntkanvtkpk rcsgsicygt iavivfflig
fmigylgyck gvepktecer lagtespvre epgedfpaar
rlywddlkrk lsekldstdf tgtikllnen syvpreagsq
kdenlalyve nqfrefklsk vwrdqhfvki qvkdsaqnsv
iivdkngrlv ylvenpggyv ayskaatvtg klvhanfgtk
kdfedlytpv ngsivivrag kitfaekvan aeslnaigvl
iymdqtkfpi vnaelsffgh ahlgtgdpyt pgfpsfnhtq
fppsrssglp nipvqtisra aaeklfgnme gdcpsdwktd
stcrmvtses knvkltvsnv lkeikilnif gvikgfvepd
hyvvvgaqrd awgpgaaksg vgtalllkla qmfsdmvlkd
gfqpsrsiif aswsagdfgs vgatewlegy lsslhlkaft
yinldkavlg tsnfkvsasp llytliektm qnvkhpvtgq
flyqdsnwas kvekltldna afpflaysgi paysfcfced
tdypylgttm dtykelieri pelnkvaraa aevagqfvik
lthdvelnld yerynsqlls fvrdlnqyra dikemglslq
wlysargdff ratsrlttdf gnaektdrfv mkklndrvmr
veyhflspyv spkespfrhv fwgsgshtlp allenlklrk
qnngafnetl frnqlalatw tiqgaanals gdvwdidnef

Preferably, the immunogen sequence used to create the at least one antibody or antigen binding fragment against the transferrin receptor comprises or consists of SEQ ID No:4 or a variant or fragment thereof.

In one embodiment, the biospecific agent may comprise at least one antibody or antigen binding fragment thereof with immunospecificity for a transferrin receptor, and at least one antibody or antigen binding fragment thereof with immunospecificity for an amyloidogenic peptide. Preferably, the biospecific agent comprises a plurality of antibodies or antigen binding fragments thereof with immunospecificity for a transferrin receptor, and a plurality of antibodies or antigen binding fragments thereof with immunospecificity for an amyloidogenic peptide.

The at least one antibody may be a whole antibody (i.e. immunoglobulin), or it may be an antigen-binding fragment or region of the corresponding full-length antibody. The at least one antibody or antigen-binding fragment thereof may be monovalent, divalent or polyvalent. Monovalent antibodies are dimers (HL) comprising a heavy (H) chain associated by a disulphide bridge with a light chain (L). Divalent antibodies are tetramer (H2L2) comprising two dimers associated by at least one disulphide bridge. Polyvalent antibodies may also be produced, for example by linking multiple dimers.

The basic structure of an antibody molecule consists of two identical light chains and two identical heavy chains which associate non-covalently and can be linked by disulphide bonds. Each heavy and light chain contains an amino-terminal variable region of about 110 amino acids, and constant sequences in the remainder of the chain. The variable region includes several hypervariable regions, or Complementarity Determining Regions (CDRs), that form the antigen-binding site of the antibody molecule and determine its specificity for the antigen, the transferrin receptor or the amyloidogenic peptide, or variant or fragment thereof (e.g. an epitope). On either side of the CDRs of the heavy and light chains is a framework region, a relatively conserved sequence of amino acids that anchors and orients the CDRs. Antibody fragments may include a bi-specific antibody (BsAb) or a chimeric antigen receptor (CAR).

The constant region consists of one of five heavy chain sequences (μ, γ, ζ, α, or ε) and one of two light chain sequences (κ or λ). The heavy chain constant region sequences determine the isotype of the antibody and the effector functions of the molecule. Preferably, the antibody or antigen-binding fragment thereof is isolated or purified.

In one embodiment, the at least one antibody or antigen-binding fragment thereof comprises a polyclonal antibody, or an antigen-binding fragment thereof. The at least one antibody or antigen-binding fragment thereof may be generated in a rabbit, mouse or rat.

However, in a preferred embodiment, the at least one antibody or antigen-binding fragment thereof comprises a monoclonal antibody or an antigen-binding fragment thereof. Preferably, the antibody of the invention is a human antibody.

As used herein, the term “human antibody” can mean an antibody, such as a monoclonal antibody, which comprises substantially the same heavy and light chain CDR amino acid sequences as found in a particular human antibody exhibiting immunospecificity for the transferrin receptor (preferably, SEQ ID No:4) or the amyloidogenic peptide (preferably, SEQ ID No:1, 2 or 3). An amino acid sequence, which is substantially the same as a heavy or light chain CDR, exhibits a considerable amount of sequence identity when compared to a reference sequence. Such identity is definitively known or recognizable as representing the amino acid sequence of the particular human antibody. Substantially the same heavy and light chain CDR amino acid sequence can have, for example, minor modifications or conservative substitutions of amino acids. Such a human antibody maintains its function of selectively binding to the transferrin receptor (preferably, SEQ ID No:4) or the amyloidogenic peptide (preferably, SEQ ID No:1, 2 or 3).

The term “human monoclonal antibody” can include a monoclonal antibody with substantially or entirely human CDR amino acid sequences produced, for example by recombinant methods such as production by a phage library, by lymphocytes or by hybridoma cells.

The term “humanised antibody” can mean an antibody from a non-human species (e.g. mouse or rabbit) whose protein sequences have been modified to increase their similarity to antibodies produced naturally in humans.

The antibody may be a recombinant antibody. The term “recombinant human antibody” can include a human antibody produced using recombinant DNA technology.

The term “antigen-binding region” can mean a region of the antibody having specific binding affinity for its target antigen, for example, the transferrin receptor or the amyloidogenic peptide. The binding region may be a hypervariable CDR or a functional portion thereof. The term “functional portion” of a CDR can mean a sequence within the CDR which shows specific affinity for the target antigen, i.e. the transferrin receptor or the amyloidogenic peptide. The functional portion of a CDR may comprise a ligand which specifically binds to the transferrin receptor or the amyloidogenic peptide.

The term “CDR” can mean a hypervariable region in the heavy and light variable chains. There may be one, two, three or more CDRs in each of the heavy and light chains of the antibody. Normally, there are at least three CDRs on each chain which, when configured together, form the antigen-binding site, i.e. the three-dimensional combining site with which the antigen binds or specifically reacts. It has however been postulated that there may be four CDRs in the heavy chains of some antibodies.

The definition of CDR also includes overlapping or subsets of amino acid residues when compared against each other. The exact residue numbers which encompass a particular CDR or a functional portion thereof will vary depending on the sequence and size of the CDR. Those skilled in the art can routinely determine which residues comprise a particular CDR given the variable region amino acid sequence of the antibody.

The term “functional fragment” of an antibody can mean a portion of the antibody which retains a functional activity. A functional activity can be, for example antigen binding activity or specificity. A functional activity can also be, for example, an effector function provided by an antibody constant region. The term “functional fragment” is also intended to include, for example, fragments produced by protease digestion or reduction of a human monoclonal antibody and by recombinant DNA methods known to those skilled in the art. Human monoclonal antibody functional fragments include, for example individual heavy or light chains and fragments thereof, such as VL, VH and Fd; monovalent fragments, such as Fv, Fab, and Fab′; bivalent fragments such as F(ab′)₂; single chain Fv (scFv); and Fc fragments.

The term “VL fragment” can mean a fragment of the light chain of a human monoclonal antibody which includes all or part of the light chain variable region, including the CDRs. A VL fragment can further include light chain constant region sequences.

The term “VH fragment” can means a fragment of the heavy chain of a human monoclonal antibody which includes all or part of the heavy chain variable region, including the CDRs.

The term “Fd fragment” can mean the light chain variable and constant regions coupled to the heavy chain variable and constant regions, i.e. VL, CL and VH, CH-1.

The term “Fv fragment” can mean a monovalent antigen-binding fragment of a human monoclonal antibody, including all or part of the variable regions of the heavy and light chains, and absent of the constant regions of the heavy and light chains. The variable regions of the heavy and light chains include, for example, the CDRs. For example, an Fv fragment includes all or part of the amino terminal variable region of about 110 amino acids of both the heavy and light chains.

The term “Fab fragment” can mean a monovalent antigen-binding fragment of a human monoclonal antibody that is larger than an Fv fragment. For example, a Fab fragment includes the variable regions, and all or part of the first constant domain of the heavy and light chains. Thus, a Fab fragment additionally includes, for example, amino acid residues from about 110 to about 220 of the heavy and light chains.

The term “Fab′ fragment” can mean a monovalent antigen-binding fragment of a human monoclonal antibody that is larger than a Fab fragment. For example, a Fab′ fragment includes all of the light chain, all of the variable region of the heavy chain, and all or part of the first and second constant domains of the heavy chain. For example, a Fab′ fragment can additionally include some or all of amino acid residues 220 to 330 of the heavy chain. Therefore, in one preferred embodiment, the at least one antibody or antigen binding fragment thereof comprises an Fab′ fragment which is immunospecific for a transferrin receptor. In another preferred embodiment, the at least one antibody or antigen binding fragment thereof comprises an Fab′ fragment which is immunospecific for an amyloidogenic peptide. Preferably, the Fab′ fragment binds specifically to oligomers and fibrils of amyloidogenic peptide, and not to amyloidogenic peptide plaques or monomers.

The term “F(ab′)₂ fragment” can mean a bivalent antigen-binding fragment of a human monoclonal antibody. An F(ab′)₂ fragment includes, for example, all or part of the variable regions of two heavy chains- and two light chains, and can further include all or part of the first constant domains of two heavy chains and two light chains.

Accordingly, in a most preferred embodiment, the at least one antibody or antigen binding fragment thereof comprises a bivalent or bispecific F(ab′)₂ fragment which is immunospecific for an amyloidogenic peptide and a transferrin receptor. The bispecific F(ab′)₂ fragment preferably comprises a first Fab′ fragment exhibiting immunospecificity to a transferrin receptor which is conjugated (preferably via its exposed sulfhydryl groups) to a second Fab′ fragment exhibiting immunospecificity to an amyloidogenic peptide. Most preferably, the second Fab′ fragment binds specifically to oligomers and fibrils of amyloidogenic peptide, and not to amyloidogenic peptide plaques or monomers.

The term “single chain Fv (scFv)” can mean a fusion of the variable regions of the heavy (VH) and light chains (VL) connected with a short linker peptide.

The term “bispecific antibody (BsAb)” can mean a bispecific antibody comprising two scFv linked to each other by a shorter linked peptide.

One skilled in the art knows that the exact boundaries of a fragment of an antibody are not important, so long as the fragment maintains a functional activity. Using well-known recombinant methods, one skilled in the art can engineer a polynucleotide sequence to express a functional fragment with any endpoints desired for a particular application. A functional fragment of the antibody may comprise or consist of a fragment with substantially the same heavy and light chain variable regions as the human antibody.

Preferably, the antigen-binding fragment thereof, with respect to the first aspect of the invention, is the transferrin receptor or the amyloidogenic peptide. The antigen-binding fragment thereof may comprise or consist of any of the fragments selected from a group consisting of VH, VL, Fd, Fv, Fab, Fab′, scFv, F (ab′)₂ and Fc fragment.

The antigen-binding fragment thereof may comprise or consist of any one of the antigen binding region sequences of the VL, any one of the antigen binding region sequences of the VH, or a combination of VL and VH antigen binding regions of a human antibody. The appropriate number and combination of VH and VL antigen binding region sequences may be determined by those skilled in the art depending on the desired affinity and specificity and the intended use of the antigen-binding fragment. Functional fragments or antigen-binding fragments of antibodies may be readily produced and isolated using methods well known to those skilled in the art. Such methods include, for example, proteolytic methods, recombinant methods and chemical synthesis. Proteolytic methods for the isolation of functional fragments comprise using human antibodies as a starting material. Enzymes suitable for proteolysis of human immunoglobulins may include, for example, papain, and pepsin. The appropriate enzyme may be readily chosen by one skilled in the art, depending on, for example, whether monovalent or bivalent fragments are required. For example, papain cleavage results in two monovalent Fab′ fragments that bind antigen and an Fc fragment. Pepsin cleavage, for example, results in a bivalent F (ab′) fragment. An F (ab′)₂ fragment of the invention may be further reduced using, for example, DTT or 2-mercaptoethanol to produce two monovalent Fab′ fragments.

Functional or antigen-binding fragments of antibodies produced by proteolysis may be purified by affinity and column chromatographic procedures. For example, undigested antibodies and Fc fragments may be removed by binding to protein A. Additionally, functional fragments may be purified by virtue of their charge and size, using, for example, ion exchange and gel filtration chromatography. Such methods are well known to those skilled in the art.

The at least one antibody or antigen-binding fragment thereof may be produced by recombinant methodology. Preferably, one initially isolates a polynucleotide encoding desired regions of the antibody heavy and light chains. Such regions may include, for example, all or part of the variable region of the heavy and light chains. Preferably, such regions can particularly include the antigen binding regions of the heavy and light chains, preferably the antigen binding sites, most preferably the CDRs.

The polynucleotide encoding the antibody or antigen-binding fragment thereof according to the invention may be produced using methods known to those skilled in the art. The polynucleotide encoding the antibody or antigen-binding fragment thereof may be directly synthesized by methods of oligonucleotide synthesis known in the art. Alternatively, smaller fragments may be synthesized and joined to form a larger functional fragment using recombinant methods known in the art.

As used herein, the term “immunospecificity” can mean the binding region is capable of immunoreacting with the amyloidogenic peptide, by specifically binding therewith. The antibody or antigen-binding fragment thereof can selectively interact with an antigen (e.g. SEQ ID No:1, 2, or 3, or a variant or fragment thereof) with an affinity constant of approximately 10⁻⁵ to 10⁻¹³ M, preferably 10⁻⁶ to 10⁻⁹ M, even more preferably, 10⁻¹⁰ to 10⁻¹² M.

As used herein, the term “immunospecificity” can mean the binding region is capable of immunoreacting with the transferrin receptor, by specifically binding therewith. Preferably, the binding affinity with the transferrin receptor is in the micromolar range. The antibody or antigen-binding fragment thereof can selectively interact with an antigen (e.g. SEQ ID No:4, or a variant or fragment thereof) with an affinity constant of no less than 1×10⁻⁴ M, more preferably no less than 1×10⁻³M.

The term “immunoreact” can mean the binding region is capable of eliciting an immune response upon binding with any of SEQ ID No:1-4, or an epitope thereof.

The term “epitope” can mean any region of an antigen with the ability to elicit, and combine with, a binding region of the antibody or antigen-binding fragment thereof.

Preferably, the antibody or antigen-binding fragment thereof according to the invention specifically binds to one or more amino acid in any of SEQ ID No:1-4.

It will be appreciated that the nanoparticle component of the biospecific agent of the invention is composed of material which enables it to be visible under infrared and/or using Magnetic Resonance Imaging (MRI) and/or Computed Tomography (CT). Such nanoparticles are also known as quantum dots.

Preferably, the nanoparticle comprises an inner core which is visible under near infrared. Preferably, the core comprises cadmium or lead. Preferably, the core comprises a material selected from CdSe, CdTe, CdS, PbS and PbSe. Most preferably, the core comprises CdSe. The mean diameter of the core may be between 5 nm and 30 nm, or between 8 nm and 20 nm, preferably between 10 nm and 5 nm, and most preferably between 12 nm and 14 nm.

Preferably, the nanoparticle comprises a shell, which preferably surrounds the core, and preferably comprising cadmium or zinc, which improves and enhances the optical properties of the core whilst increasing the quantum yield (i.e. the number of photons absorbed/the number of photons emitted). The shell may comprise ZnS or CdS. Preferably, the shell comprises ZnS. Advantageously, as shown in FIG. 2, the shell also reduces the toxicity of the cadmium-based or lead-based core. The shell is preferably grown around the core, forming a layer.

The nanoparticle preferably comprises a contrast material, which is visible using MRI or CT. Preferably, the contrast material encapsulates or surrounds the core, and more preferably the shell.

The contrast material may comprise a metallic or non-metallic material. The contrast material may comprise a magnetic or non-magnetic material. In embodiments where the contrast material is magnetic, it may comprise an MRI contrast material. The contrast material may comprise a paramagnetic or superparamagnetic material. For example, the contrast material core may comprise iron, nickel, cobalt or dysprosium or a compound, such as an oxide or alloy, which contains one or more of these elements. The contrast material may comprise magnetite (Fe₃O₄).

In embodiments wherein the contrast material is non-magnetic, it may comprise both a MRI and a CT contrast material. For example, the contrast material may comprise gadolinium, gold, iodine or boro-sulphate. Each of these materials may be used as either MRI or as CT contrast materials. Preferably, the contrast material comprises gadolinium.

Preferably, the contrast material comprises 1,4,7,10-tetraazacyclododecane-1,4,7,10-tetraacetic acid (i.e. DOTA). Most preferably, the contrast material comprises gadoteric acid, a macrocycle-structured Gd-based MRI contrast agent, consisting of the organic acid “DOTA” as a chelating agent.

The contrast material preferably encapsulates the shell. The Gd-DOTA/silica encapsulation may be attached to the shell by means of surface salinization. 3-Mercaptopropyl trimethoxysilane (MPS) can act as a primer to the surface by means of Zn/thiol structures. Present methoxysilane groups (Si—OCHB3B) can hydrolyze into silanol groups (Si—OH) and therefore cross-link, which stabilizes the silane layer onto the surface of the shell. The addition of sodium silicate and hydrophilic trimethoxysilane allows the cross-linking of trimethoxysilane groups by means of the formation of siloxane bonds, which ensure that the silica layer is connected with the primer layer and ultimately to the shell.

The at least one antibody or antigen binding fragment thereof may be attached to the contrast agent of the biospecific agent by covalent bonding. Preferably, the contrast agent is configured to allow for carboxyl functionalization by which the at least one antibody or antigen fragment thereof may be conjugated thereto. In one embodiment, the at least one antibody or antigen binding fragment thereof may be covalently attached to the contrast agent using carbodiimide chemistry in order to create the biospecific agent of the invention. For example, as described in the examples, EDC (1-ethyl-3-(3-dimethylamino) propyl carbodiimide hydrochloride) is a water-soluble carbodiimide crosslinker that activates carboxyl groups for spontaneous reaction with primary amines, enabling antibody immobilisation and hapten-carrier protein conjugation. Thus, conjugation involved covalent bonding of the antibody's amine group to carboxyl groups present on the contrast agent outer layer.

The amount of antibody or antigen binding fragment thereof that is attached to the nanoparticle depends on the amount of functional groups (preferably carboxyl groups) on the contrast material, the type of contrast material and the chemistry of attachment. Preferably, a plurality of antibodies or antigen binding fragments thereof are arranged in a spaced-apart array covering the outer surface of the contrast material layer.

The biospecific agent may be substantially spherical in shape. The mean diameter of the biospecific agent may be sub-micron, i.e. less than 1000 nm, more preferably less than 500 nm, or even more preferably less than 300 nm. The mean diameter of the biospecific agent may be 100-450 nm.

Accordingly, in one preferred embodiment, the biospecific agent comprises a nanoparticle comprising:

(i) a CdSe, CdTe, CdS, PbS or PbSe inner core (preferably, CdSe), which is visible under near infrared;
(ii) a zinc or cadmium shell surrounding the core (preferably, ZnS), which improves and enhances the optical properties of the core whilst increasing the quantum yield;
(iii) a contrast material encapsulating the shell (preferably, Gd-DOTA/silica), visible using Magnetic Resonance Imaging (MRI) and/or Computed Tomography (CT); and
(iv) at least one antibody or antigen binding fragment thereof, which is immunospecific for a transferrin receptor and an amyloidogenic peptide.

Preferably, the at least one antibody or antigen binding fragment thereof comprises a bispecific F(ab′)₂ fragment which is immunospecific for an amyloidogenic peptide and a transferrin receptor. The bispecific F(ab′)₂ fragment preferably comprises a first Fab′ fragment exhibiting immunospecificity to a transferrin receptor and a second Fab′fragment exhibiting immunospecificity to an amyloidogenic peptide. Most preferably, the second Fab′ fragment binds specifically to the oligomers and fibrils of the amyloidogenic peptide, but not to amyloidogenic peptide plaques or monomers.

As described in the Examples, the inventor has demonstrated that the biospecific agent of the invention has utility in both diagnosis and therapy of neurodegenerative disorders.

Thus, in a second aspect, there is provided an amyloidogenic peptide biospecific agent according to the first aspect, for use in diagnosis.

It will be appreciated that the biospecific agent may be used as a biosensor in a range of different biological imaging applications. For example, the biospecific agent is preferably used in MRI, CT or IR imaging techniques, as a biolabel.

Thus, in a third aspect, there is provided use of the amyloidogenic peptide biospecific agent of the first aspect, as an NIR biolabel, an MRI biolabel or as a CT biolabel.

In a fourth aspect, there is provided a biolabel comprising the amyloidogenic peptide biospecific agent according to the first aspect.

The biolabel may be used in NIR, MRI or CT imaging.

In a fifth aspect, therefore, there is provided an NIR, MRI or CT imaging method comprising the use of the amyloidogenic peptide biospecific agent of the first aspect.

Preferably, the biospecific agents of the invention emit light in the near infrared region. Infrared is defined as radiation of wavelength 700 nm to 1 mm, and near infrared has a wavelength of about 0.75-1.4 μm. Near infrared I, which is most preferred, has a wavelength of about 705 nm-900 nm. Preferably the biospecific agent of the invention results in reduced autofluorescence with increase photoluminescence with the ability of non-invasive detection via functional NIR I spectroscopy. The maximum emission wavelength of the agent of the invention is at around 850 nm with the maximum absorption wavelength being at 496 nm. Advantageously, due to the use of quantum dots, the emission peak, being in the NIR, can penetrate through biological tissue to more than 2 nm, which is ideal for diagnosis of brain diseases, such as Alzheimer's disease.

Preferably, the biospecific agent of the invention has MRI and/or CT detection properties due to the Gd-DOTA silica. Magnetic Resonance Imaging (MRI) and Computed Tomography (CT) are the methods of choice in the imaging of tissues. MRI is based on the ability of large magnetic fields to produce a net magnetic vector temporarily changing the alignment of the protons in the highly hydrated tissues. MRI is mainly suited for the imaging of injuries in ligaments, tendons and spinal cord as well as of brain tumours. However, the technique does not allow imaging of brain disorders as detailed as those that can be obtained by CT.

CT is based on X-ray attenuation which is detected by a detector where the value of pixels is calculated and then transformed into an image. Quantitative computed tomography (QCI) is able to provide measurements of brain density, and measures the true volumetric (mg/cm³) in three dimensions, as opposed to the two dimensional area of brain density.

The inventors have demonstrated that the amyloidogenic peptide biospecific agent according to the invention can be used in imaging symptoms of neurodegenerative disorders due to the common presence of an amyloidogenic peptide.

For example, the neurodegenerative disorder may be selected from a group consisting of Alzheimer's disease; Parkinson's disease; Huntington's disease; Motor Neurone disease; Spinocerebellar type 1, type 2, and type 3; Amyotrophic Lateral Sclerosis (ALS); and Frontotemporal Dementia. Preferably, the disorder is Alzheimer's disease.

It will be appreciated that in order to diagnose Alzheimer's disease, the at least one antibody or antigen binding fragment thereof preferably binds specifically to amyloid beta (preferably partially aggregated SEQ ID No:1) oligomers and fibrils, but not to amyloid plaques. In order to diagnose Huntington's disease, the at least one antibody or antigen binding fragment thereof preferably binds specifically to Huntingtin (preferably partially aggregated SEQ ID No:2) oligomers and fibrils, but not to Huntingtin plaques and monomers. In order to diagnose Parkinson's disease, the at least one antibody or antigen binding fragment thereof preferably binds specifically to alpha-synuclein (preferably partially aggregated SEQ ID No:3) oligomers and fibrils, but not to alpha-synuclein plaques and monomers. In each of the embodiments described herein, the biospecific agent comprises at least one antibody or antigen binding fragment which is immunospecific for a transferrin receptor (preferably SEQ ID No:4), because this enables crossing of the blood brain barrier.

The biospecific agent according to the first aspect may be used in in vivo, ex vivo or in vitro diagnosis.

The invention also provides a kit for diagnosing patients suffering from neurodegenerative disease.

Hence, according to a sixth aspect of the invention, there is provided a kit for diagnosing a subject suffering from a neurodegenerative disorder, or a pre-disposition thereto, or for providing a prognosis of the subject's condition, the kit comprising the biospecific agent according to the first aspect configured to detect the concentration of amyloidogenic peptide present in a biological sample from a test subject, wherein presence of peptide in the sample suggests that the subject suffers from neurodegenerative disorder.

According to a seventh aspect, there is provided a method for diagnosing a subject suffering from neurodegenerative disorder, or a pre-disposition thereto, or for providing a prognosis of the subject's condition, the method comprising detecting the concentration of amyloidogenic peptide present in a biological sample obtained from a subject, wherein the detection is achieved using the biospecific agent according to the first aspect, and wherein presence of antigen in the sample suggests that the subject suffers from neurodegenerative disorder.

The sample may comprise blood, urine, tissue, brain biopsy etc.

Preferably, the kit or method is used to identify the presence or absence of amyloidogenic peptide oligomers and fibrils (including protofibrils) in the sample (but not amyloidogenic peptide plaques and monomers), as oligomers and fibrils are indicative of early stage neurodegenerative disorder, or determine the concentration thereof in the sample. The detection means may comprise an assay adapted to detect the presence and/or absence of the amyloidogenic peptide in the sample. The kit or method may comprise the use of a positive control and/or a negative control against which the assay may be compared. For example, the kit may comprise a reference for the concentration of amyloidogenic peptide in a sample from an individual who does (i.e. positive control) or does not (i.e. a negative control) suffer from neurodegenerative disorder.

The kit may further comprise a label which may be detected. The term “label” can mean a moiety that can be attached to the biospecific agent. Moieties can be used, for example, for therapeutic or diagnostic procedures. Therapeutic labels include, for example, moieties that can be attached to the agent of the invention and used to monitor the binding of the agent to the amyloidogenic peptide. Diagnostic labels include, for example, moieties which can be detected by analytical methods. Analytical methods include, for example, qualitative and quantitative procedures. Qualitative analytical methods include, for example, immunohistochemistry and indirect immunofluorescence. Quantitative analytical methods include, for example, immunoaffinity procedures such as radioimmunoassay, ELISA or FACS analysis. Analytical methods also include both in vitro and in vivo imaging procedures. Specific examples of diagnostic labels that can be detected by analytical means include enzymes, radioisotopes, fluorochromes, chemiluminescent markers, and biotin. A label can be attached directly to the agent of the invention, or be attached to a secondary binding agent that specifically binds the agent of the invention. Such a secondary binding agent can be, for example, a secondary antibody. A secondary antibody can be either polyclonal or monoclonal, and of human, rodent or chimeric origin.

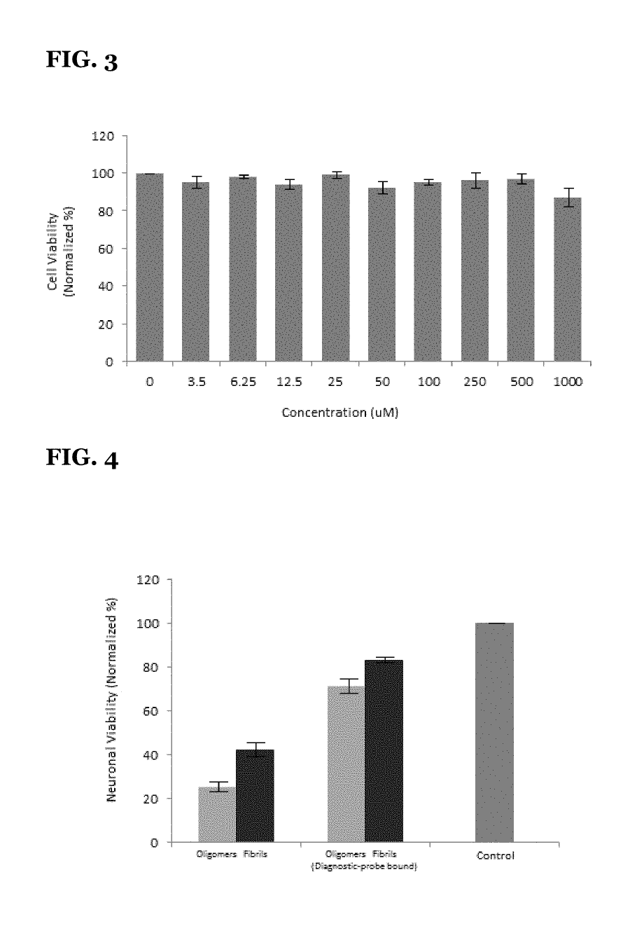

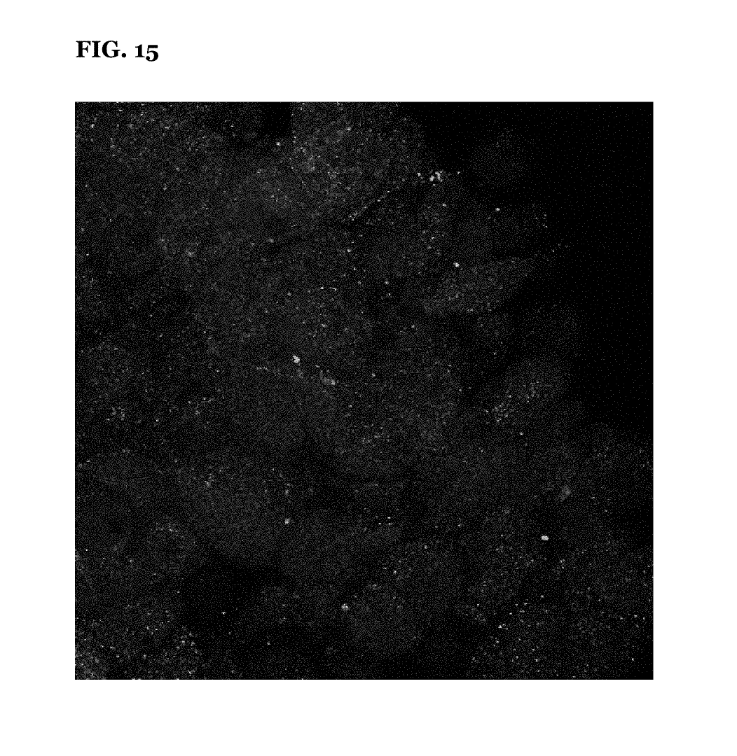

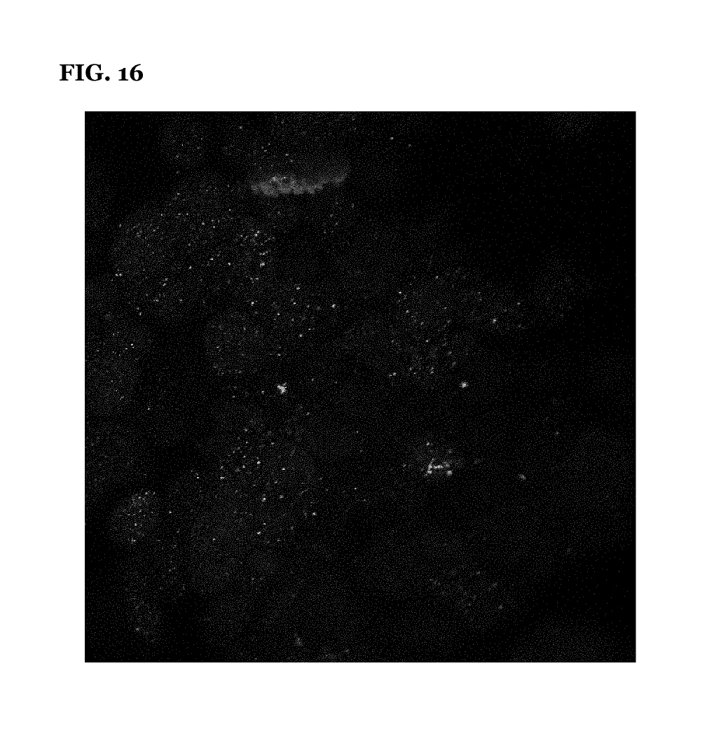

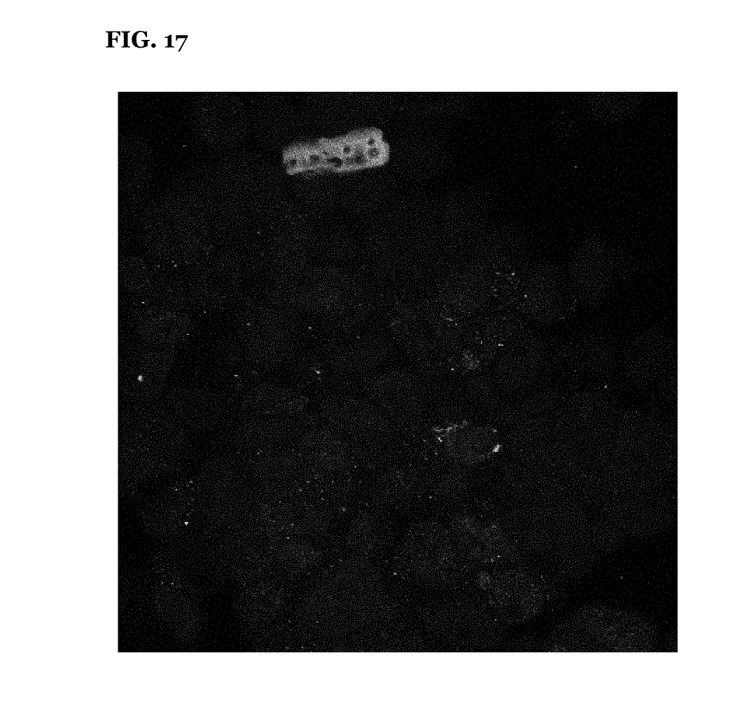

In addition to the various imaging and diagnostic techniques that can harness the powerful amyloidogenic-targeting properties of the biospecific agent, the examples and FIGS. 3, 14-17 also explain how the conjugated bispecific antibody or antigen binding fragment thereof which specifically targets the amyloidogenic peptide renders the neurotoxic amyloid-beta oligomers insoluble, as they are immobilised. As such, these bound oligomers are less readily able to enter neuron cells and their toxicity is significantly reduced. This shows that the biospecific agent has significant therapeutic potential by binding to the neurotoxic amyloid-beta oligomers and inhibiting them from entering cells.

Therefore, according to a eighth aspect, there is provided the amyloidogenic peptide biospecific agent according to the first aspect, for use in therapy.

The biospecific agent of the invention is particularly useful for preventing or treating neurodegenerative disorders.

Hence, in a ninth aspect, there is provided an amyloidogenic peptide biospecific agent according to the first aspect, for use in treating, ameliorating or preventing a neurodegenerative disorder.

In a tenth aspect, there is provided a method of treating, ameliorating or preventing a neurodegenerative disorder in a subject, the method comprising, administering to a subject in need of such treatment, a therapeutically effective amount of an amyloidogenic peptide biospecific agent according to the first aspect.

Preferably, the neurodegenerative disorder is selected from a group consisting of Alzheimer's disease; Parkinson's disease; Huntington's disease; Motor Neurone disease; Spinocerebellar type 1, type 2, and type 3; Amyotrophic Lateral Sclerosis (ALS); and Frontotemporal Dementia, and is preferably Alzheimer's disease.

The neurodegenerative disorder which is treated is preferably which is characterised by the damage or death of ‘Global’ neurons. For example, the neurodegenerative disorder may be selected from a group consisting of Alzheimer's disease; Parkinson's disease; Huntington's disease; Motor Neurone disease; Spinocerebellar type 1, type 2, and type 3; Amyotrophic Lateral Sclerosis (ALS); and Frontotemporal Dementia.

Preferably, the neurodegenerative disorder, which is treated, is Alzheimer's disease, Parkinson's disease, Huntington's disease or Motor Neurone disease. Most preferably, the neurodegenerative disorder, which is treated, is Alzheimer's disease.

It will be appreciated that biospecific agents according to the invention may be used in a monotherapy (e.g. the use of an antibody or antigen binding fragment thereof alone, or the use of the antibody-drug conjugate alone), for treating, ameliorating or preventing a neurodegenerative disorder. Alternatively, agents according to the invention may be used as an adjunct to, or in combination with, known therapies for treating, ameliorating, or preventing neurodegenerative disorders, such as such as other acetylcholinesterase inhibitors.

The agents according to the invention may be combined in compositions having a number of different forms depending, in particular, on the manner in which the composition is to be used. Thus, for example, the composition may be in the form of a powder, tablet, capsule, liquid, ointment, cream, gel, hydrogel, aerosol, spray, micellar solution, transdermal patch, liposome suspension or any other suitable form that may be administered to a person or animal in need of treatment. It will be appreciated that the vehicle of medicaments according to the invention should be one which is well-tolerated by the subject to whom it is given, and preferably enables delivery of the agents across the blood-brain barrier.

Medicaments comprising agents of the invention may be used in a number of ways. For instance, oral administration may be required, in which case the agents may be contained within a composition that may, for example, be ingested orally in the form of a tablet, capsule or liquid. Compositions comprising agents and medicaments of the invention may be administered by inhalation (e.g. intranasally). Compositions may also be formulated for topical use. For instance, creams or ointments may be applied to the skin, for example adjacent to the brain.

Agents and medicaments according to the invention may also be incorporated within a slow- or delayed-release device. Such devices may, for example, be inserted on or under the skin, and the medicament may be released over weeks or even months. The device may be located at least adjacent the treatment site, i.e. the brain. Such devices may be particularly advantageous when long-term treatment with agents used according to the invention is required and which would normally require frequent administration (e.g. at least daily injection).

In a preferred embodiment, agents and medicaments according to the invention may be administered to a subject by injection into the blood stream or directly into a site requiring treatment. For example, the medicament may be injected at least adjacent the brain. Injections may be intravenous (bolus or infusion) or subcutaneous (bolus or infusion), or intradermal (bolus or infusion).

It will be appreciated that the amount of the biospecific agent that is required is determined by its biological activity and bioavailability, which in turn depends on the mode of administration, the physiochemical properties of the agent, and whether it is being used as a monotherapy or in a combined therapy. The frequency of administration will also be influenced by the half-life of the agent within the subject being treated. Optimal dosages to be administered may be determined by those skilled in the art, and will vary with the particular agent in use, the strength of the pharmaceutical composition, the mode of administration, and the advancement of the bacterial infection. Additional factors depending on the particular subject being treated will result in a need to adjust dosages, including subject age, weight, gender, diet, and time of administration.

Generally, a daily dose of between 0.001 μg/kg of body weight and 10 mg/kg of body weight of agent according to the invention may be used for treating, ameliorating, or preventing neurodegenerative disorder, depending upon which agent. More preferably, the daily dose of agent is between 0.01 μg/kg of body weight and 1 mg/kg of body weight, more preferably between 0.1 μg/kg and 100 μg/kg body weight, and most preferably between approximately 0.1 μg/kg and 10 μg/kg body weight.

The agent may be administered before, during or after onset of a neurodegenerative disorder. Daily doses may be given as a single administration (e.g. a single daily injection). Alternatively, the agent may require administration twice or more times during a day. As an example, agents may be administered as two (or more depending upon the severity of the neurodegenerative disorder being treated) daily doses of between 0.07 μg and 700 mg (i.e. assuming a body weight of 70 kg). A patient receiving treatment may take a first dose upon waking and then a second dose in the evening (if on a two dose regime) or at 3- or 4-hourly intervals thereafter. Alternatively, a slow release device may be used to provide optimal doses of agents according to the invention to a patient without the need to administer repeated doses. Known procedures, such as those conventionally employed by the pharmaceutical industry (e.g. in vivo experimentation, clinical trials, etc.), may be used to form specific formulations of the agents according to the invention and precise therapeutic regimes (such as daily doses of the agents and the frequency of administration).

In an eleventh aspect of the invention, there is provided a pharmaceutical composition comprising a biospecific agent according to the first aspect; and optionally a pharmaceutically acceptable vehicle.

The pharmaceutical composition is preferably an anti-neurodegenerative disease composition, i.e. a pharmaceutical formulation used in the therapeutic amelioration, prevention or treatment of a neurodegenerative disorder in a subject, such as preferably Alzheimer's disease, Parkinson's disease or Huntington's disease.

The invention also provides in a twelfth aspect, a process for making the pharmaceutical composition according to the ninth aspect, the process comprising combining a therapeutically effective amount of a biospecific agent according to the first aspect with a pharmaceutically acceptable vehicle.

The at least one antibody or antigen binding fragment thereof may be as defined with respect to the first aspect. Preferably, the at least one antibody or antigen binding fragment thereof comprises a bispecific F(ab′)₂ fragment which is immunospecific for an amyloidogenic peptide and a transferrin receptor. The bispecific F(ab′)₂ fragment preferably comprises a first Fab′ fragment exhibiting immunospecificity to a transferrin receptor which is conjugated to a second Fab′ fragment exhibiting immunospecificity to an amyloidogenic peptide. Most preferably, the second Fab′ fragment binds specifically for the oligomers and fibrils of amyloid beta protein, and not for amyloidogenic peptide plaques and peptide monomers.

A “subject” may be a vertebrate, mammal, or domestic animal. Hence, medicaments according to the invention may be used to treat any mammal, for example livestock (e.g. a horse), pets, or may be used in other veterinary applications. Most preferably, the subject is a human being.

A “therapeutically effective amount” of biospecific agent is any amount which, when administered to a subject, is the amount of agent that is needed to treat the neurodegenerative disease, or produce the desired effect.

For example, the therapeutically effective amount of biospecific agent used may be from about 0.001 ng to about 1 mg, and preferably from about 0.01 ng to about 100 ng. It is preferred that the amount of biospecific agent is an amount from about 0.1 ng to about 10 ng, and most preferably from about 0.5 ng to about 5 ng.

A “pharmaceutically acceptable vehicle” as referred to herein, is any known compound or combination of known compounds that are known to those skilled in the art to be useful in formulating pharmaceutical compositions.

In one embodiment, the pharmaceutically acceptable vehicle may be a solid, and the composition may be in the form of a powder or tablet. A solid pharmaceutically acceptable vehicle may include one or more substances which may also act as flavouring agents, lubricants, solubilisers, suspending agents, dyes, fillers, glidants, compression aids, inert binders, sweeteners, preservatives, dyes, coatings, or tablet-disintegrating agents. The vehicle may also be an encapsulating material. In powders, the vehicle is a finely divided solid that is in admixture with the finely divided active agents according to the invention. In tablets, the active agent may be mixed with a vehicle having the necessary compression properties in suitable proportions and compacted in the shape and size desired. The powders and tablets preferably contain up to 99% of the active agents. Suitable solid vehicles include, for example calcium phosphate, magnesium stearate, talc, sugars, lactose, dextrin, starch, gelatin, cellulose, polyvinylpyrrolidine, low melting waxes and ion exchange resins. In another embodiment, the pharmaceutical vehicle may be a gel and the composition may be in the form of a cream or the like.

However, the pharmaceutical vehicle may be a liquid, and the pharmaceutical composition is in the form of a solution. Liquid vehicles are used in preparing solutions, suspensions, emulsions, syrups, elixirs and pressurized compositions. The active agent according to the invention may be dissolved or suspended in a pharmaceutically acceptable liquid vehicle such as water, an organic solvent, a mixture of both or pharmaceutically acceptable oils or fats. The liquid vehicle can contain other suitable pharmaceutical additives such as solubilisers, emulsifiers, buffers, preservatives, sweeteners, flavouring agents, suspending agents, thickening agents, colours, viscosity regulators, stabilizers or osmo-regulators. Suitable examples of liquid vehicles for oral and parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the vehicle can also be an oily ester such as ethyl oleate and isopropyl myristate. Sterile liquid vehicles are useful in sterile liquid form compositions for parenteral administration. The liquid vehicle for pressurized compositions can be a halogenated hydrocarbon or other pharmaceutically acceptable propellant.

Liquid pharmaceutical compositions, which are sterile solutions or suspensions, can be utilized by, for example, intramuscular, intrathecal, epidural, intraperitoneal, intravenous and particularly subcutaneous injection. The agent may be prepared as a sterile solid composition that may be dissolved or suspended at the time of administration using sterile water, saline, or other appropriate sterile injectable medium.

The agents and compositions of the invention may be administered orally in the form of a sterile solution or suspension containing other solutes or suspending agents (for example, enough saline or glucose to make the solution isotonic), bile salts, acacia, gelatin, sorbitan monoleate, polysorbate 80 (oleate esters of sorbitol and its anhydrides copolymerized with ethylene oxide) and the like. The agents used according to the invention can also be administered orally either in liquid or solid composition form. Compositions suitable for oral administration include solid forms, such as pills, capsules, granules, tablets, and powders, and liquid forms, such as solutions, syrups, elixirs, and suspensions. Forms useful for parenteral administration include sterile solutions, emulsions, and suspensions.

It will be appreciated that the invention extends to any nucleic acid or peptide or variant, derivative or analogue thereof, which comprises or consists of substantially the amino acid or nucleic acid sequences of any of the sequences referred to herein, including variants or fragments thereof. The terms “substantially the amino acid/nucleotide/peptide sequence”, “variant” and “fragment”, can be a sequence that has at least 40% sequence identity with the amino acid/nucleotide/peptide sequences of any one of the sequences referred to herein, for example 40% identity with the sequence identified as SEQ ID No: 1-4, and so on.

Amino acid/polynucleotide/polypeptide sequences with a sequence identity which is greater than 50%, more preferably greater than 65%, 70%, 75%, and still more preferably greater than 80% sequence identity to any of the sequences referred to are also envisaged. Preferably, the amino acid/polynucleotide/polypeptide sequence has at least 85% identity with any of the sequences referred to, more preferably at least 90%, 92%, 95%, 97%, 98%, and most preferably at least 99% identity with any of the sequences referred to herein.

The skilled technician will appreciate how to calculate the percentage identity between two amino acid/polynucleotide/polypeptide sequences. In order to calculate the percentage identity between two amino acid/polynucleotide/polypeptide sequences, an alignment of the two sequences must first be prepared, followed by calculation of the sequence identity value. The percentage identity for two sequences may take different values depending on:—(i) the method used to align the sequences, for example, ClustalW, BLAST, FASTA, Smith-Waterman (implemented in different programs), or structural alignment from 3D comparison; and (ii) the parameters used by the alignment method, for example, local vs global alignment, the pair-score matrix used (e.g. blosum62, pam250, gonnet etc.), and gap-penalty, e.g. functional form and constants.

Having made the alignment, there are many different ways of calculating percentage identity between the two sequences. For example, one may divide the number of identities by: (i) the length of shortest sequence; (ii) the length of alignment; (iii) the mean length of sequence; (iv) the number of non-gap positions; or (iv) the number of equivalenced positions excluding overhangs. Furthermore, it will be appreciated that percentage identity is also strongly length dependent. Therefore, the shorter a pair of sequences is, the higher the sequence identity one may expect to occur by chance.

Hence, it will be appreciated that the accurate alignment of protein or DNA sequences is a complex process. The popular multiple alignment program ClustalW (Thompson et al., 1994, Nucleic Acids Research, 22, 4673-4680; Thompson et al., 1997, Nucleic Acids Research, 24, 4876-4882) is a preferred way for generating multiple alignments of proteins or DNA in accordance with the invention. Suitable parameters for ClustalW may be as follows: For DNA alignments: Gap Open Penalty=15.0, Gap Extension Penalty=6.66, and Matrix=Identity. For protein alignments: Gap Open Penalty=10.0, Gap Extension Penalty=0.2, and Matrix=Gonnet. For DNA and Protein alignments: ENDGAP=−1, and GAPDIST=4. Those skilled in the art will be aware that it may be necessary to vary these and other parameters for optimal sequence alignment.

Preferably, calculation of percentage identities between two amino acid/polynucleotide/polypeptide sequences may then be calculated from such an alignment as (N/T)*100, where N is the number of positions at which the sequences share an identical residue, and T is the total number of positions compared including gaps but excluding overhangs. Hence, a most preferred method for calculating percentage identity between two sequences comprises (i) preparing a sequence alignment using the clustalw program using a suitable set of parameters, for example, as set out above; and (ii) inserting the values of n and t into the following formula:—sequence identity=(N/T)*100.

Alternative methods for identifying similar sequences will be known to those skilled in the art. For example, a substantially similar nucleotide sequence will be encoded by a sequence which hybridizes to any of the nucleic acid sequences shown herein, or their complements under stringent conditions. By stringent conditions, we mean the nucleotide hybridises to filter-bound DNA or RNA in 3× sodium chloride/sodium citrate (SSC) at approximately 45c followed by at least one wash in 0.2×ssc/0.1% SDS at approximately 20-65° c. Alternatively, a substantially similar polypeptide may differ by at least 1, but less than 5, 10, 20, 50 or 100 amino acids from the sequences shown herein.

Due to the degeneracy of the genetic code, it is clear that any nucleic acid sequence described herein could be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent change. Other suitable variants are those having homologous nucleotide sequences but comprising all, or portions of, sequence, which are altered by the substitution of different codons that encode an amino acid with a side chain of similar biophysical properties to the amino acid it substitutes, to produce a conservative change. For example small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid. It will therefore be appreciated which amino acids may be replaced with an amino acid having similar biophysical properties, and the skilled technician will know the nucleotide sequences encoding these amino acids.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying Figures, in which:—

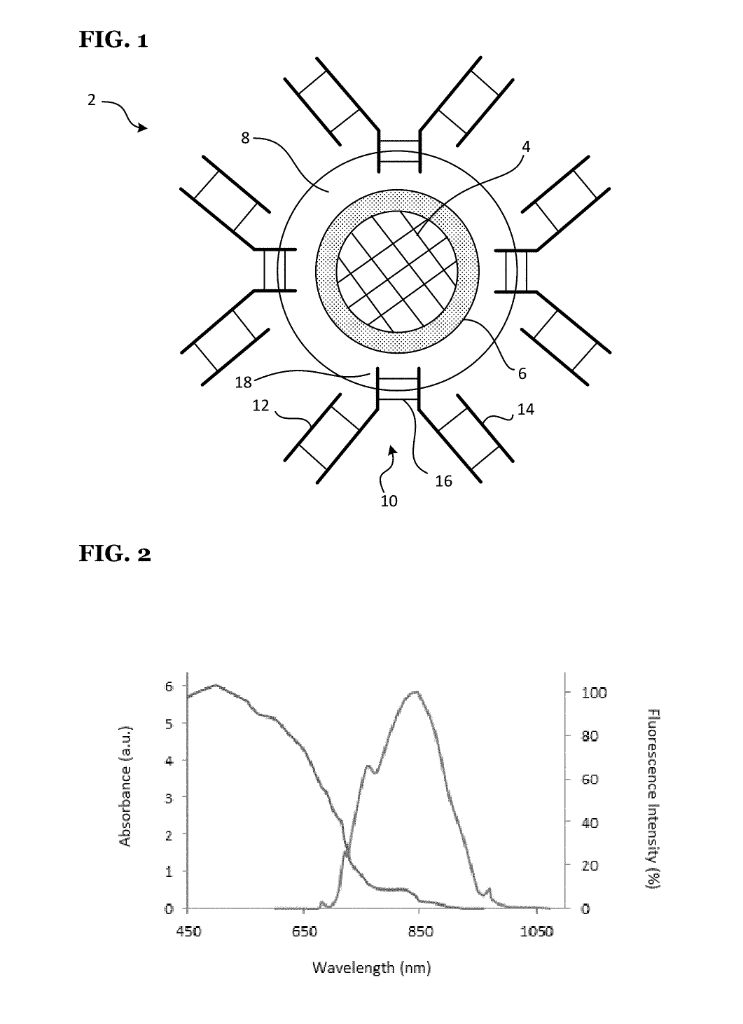

FIG. 1 is a schematic representation of one embodiment of a nanoparticle or “quantum dot” according to the invention. The nanoparticle includes a Cadmium selenide core, and a Zinc sulphide shell, which is encapsulated by a Gd-DOTA silica outer shell to which are conjugated bispecific antibodies or antigen-binding fragments thereof with immunospecificity for amyloid beta and the transferrin receptor;

FIG. 2 shows absorption and emission spectra of Gd-DOTA silica encapsulated CdSe/ZnS nanoparticles conjugated to a bispecific antibody acting as a “diagnostic probe”;

FIG. 3 is a barchart showing cell viability following exposure to the nanoparticle of the invention;

FIG. 4 is a barchart showing neuronal viability following exposure to the nanoparticle;

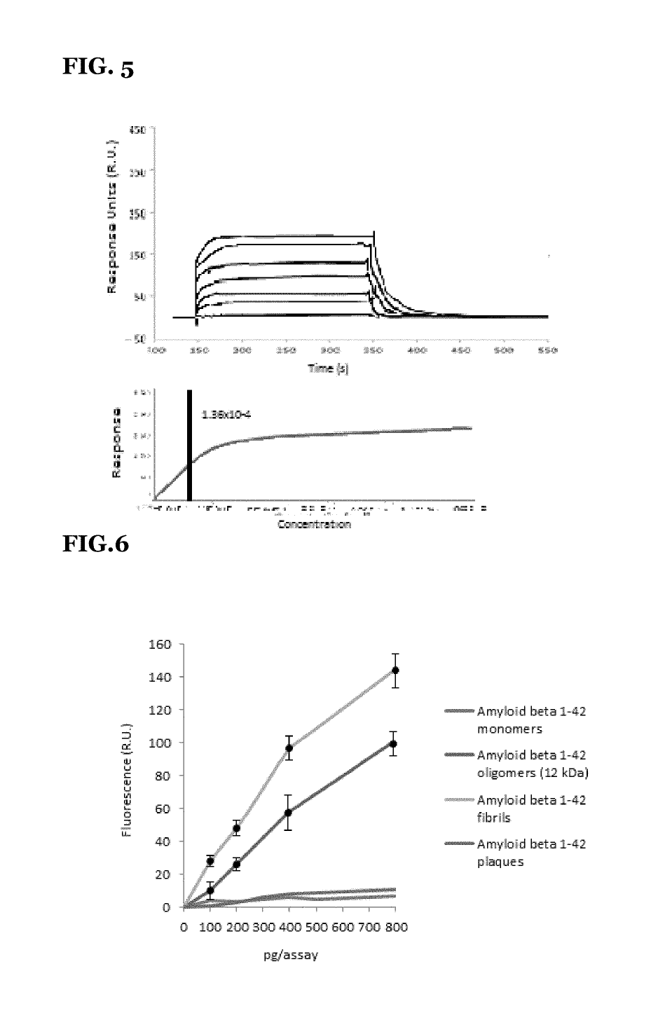

FIG. 5 shows the results of surface plasmon resonance employed to determine the affinity of the nanoparticle to transferrin receptors;

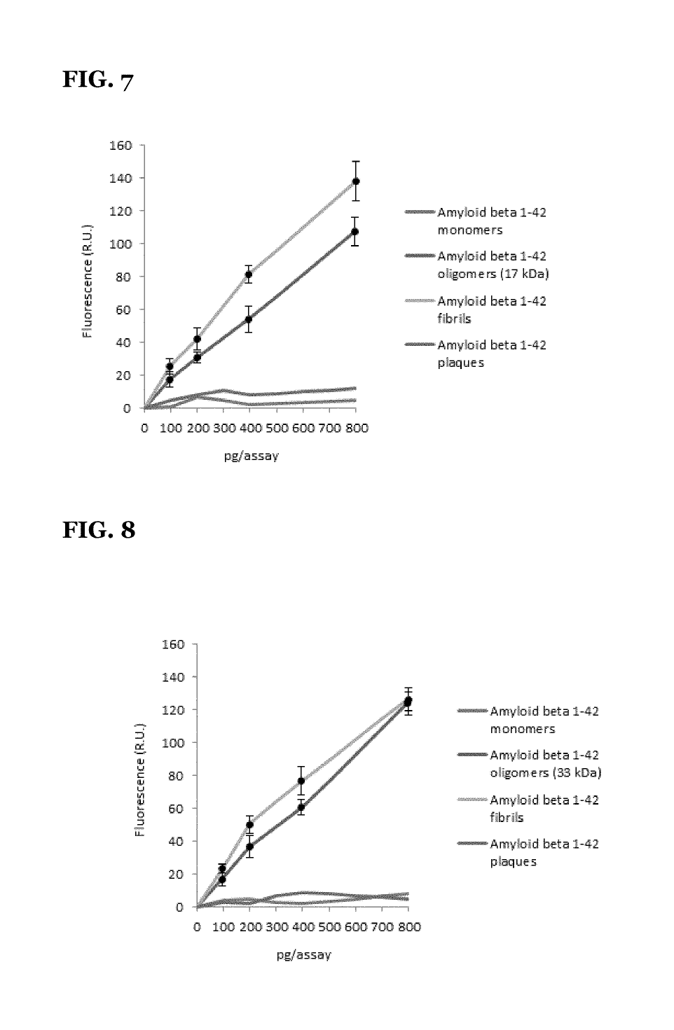

FIG. 6-11 show fluorescent data of the nanoparticle;

FIG. 12 shows the immunofluorescence staining of C57Bl/Sv129. a) Shows DAPI nuclei stain. b) Shows the localization of insulin. c) amyloid-beta oligomers;

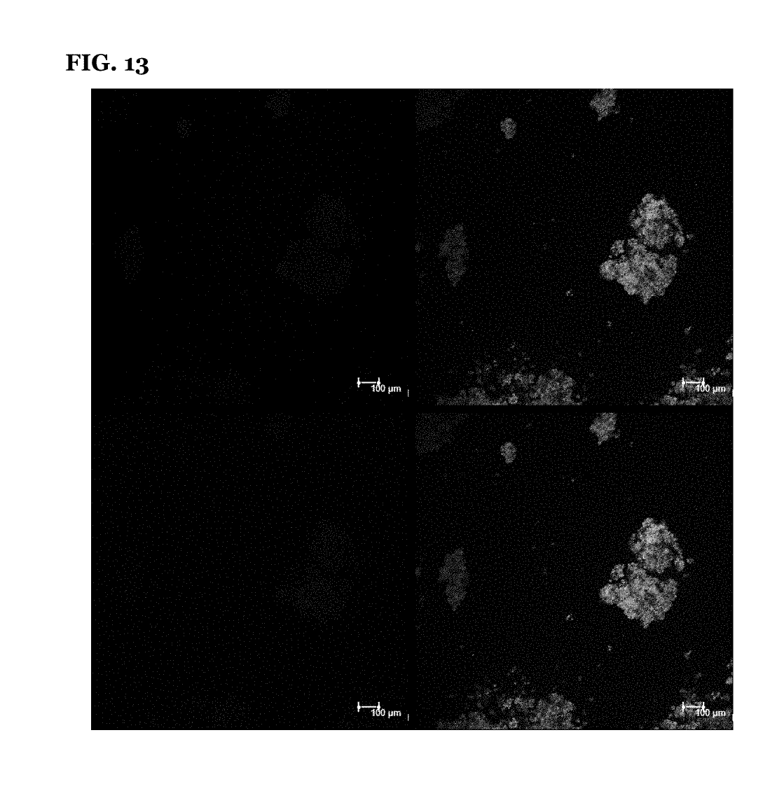

FIG. 13 shows different immunofluorescence approach was taken; and

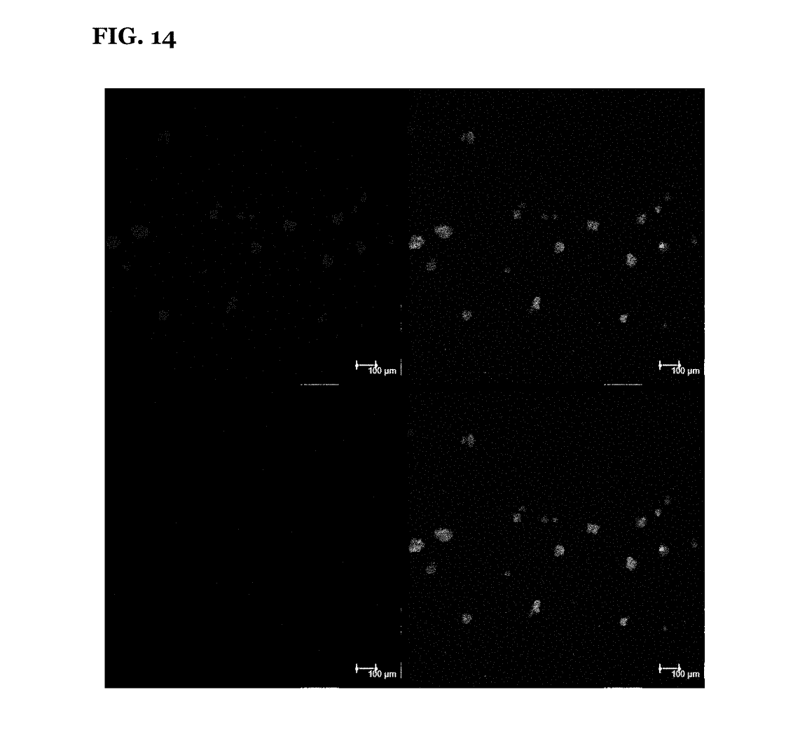

FIG. 14-17 shows the results after amyloid-beta oligomers and the nanoparticle were incubated together for 30+ minutes.

### EXAMPLES

### Materials and Methods

### 1) F(ab′)2 Fragments

F(ab′)2 fragments were obtained using commercially available kits from Life Technologies:

Monoclonal Antibody (mAB) Anti-Aβ (Oligomer and Fibril Specific) Antibody (IgG1) F(Ab′)2 Generation:

0.5 mL of the antibody (8 mg/mL) was added to a previously equilibriated immobilised ficin column and incubated (37° C.) for 25 hours. Generated F(ab′)2 fragments were purified with NAb Protein A Column and centrifuged (1000×g) for 1 minute. Flow-through concentration was determined spectrophotometrically by measuring the absorbance at 280 nm.

Monoclonal Antibody (mAB) Anti-TfR (Transferrin Receptor) Antibody (IgM) F(ab′)2 Generation:

A previously equilibriated immobilised pepsin column was washed with 8 mL IgM F(ab′)2 digestion buffer (200 ml, 100 mM sodium acetate, 150 mM NaCl, 0.05% NaN3; pH4.5). The column and 1 mL of antibody (1 mg/mL) were incubated (37° C.) separately for 3 minutes. Antibody was added to column and incubated (37° C.) for 1.5 hours. Generated F(ab′)2 fragments were centrifuged in C30 Concentrator and concentration was determined spectrophotometrically by measuring absorbance at 595 nm.

### 2) Bispecific Antibody Synthesis (Including Fab′ Generation)

Antibody synthesis was performed as described by Greg T. Hermanson in Bioconjugate Techniques Second Edition, ISBN: 978-0-12-370501-3.

### Fab′ Generation:

1 mL of anti-AB oligomer-specific antibody F(ab′)2 (10 mg/mL) was dissolved in 20 mM buffer (sodium phosphate, 0.15M NaCl, 5 mM EDTA, pH7.4). 6 mg of 2-MEA.HCl was added and incubated (37° C.) for 1.5 hours. Excess 2-MEA*HCl was removed by gel-filtration. Protocol was repeated for anti-TfR antibody Fab′ generation.

Bispecific Antibody Synthesis:

Anti-Aβ oligomer-specific antibody Fab′(Fab′A) was added to DTNB (40 mg DTNB, 10 ml 1MTris-HCl, pH7.5) and incubated at room temperature. Equimolar ratios of Fab′A-DTNB and anti-TfR antibody (Fab′B) were mixed and incubated (37° C.) for 1.5 hours. Reaction was incubated (4° C.) overnight. Bispecific BsAb fraction was purified with Superdex 200 column equilibriated in PBS.

### 3) Synthesis of CdSe/ZnS Nanoparticles

Nanoparticles were synthesised with silica encapsulation based on publications from Yang Xu et aL & B. O. Dabbousi et al. However, the protocol described herein further incorporates gadolinium in the outer shell and allows for carboxyl functionalization to allow antibody fragment conjugation.

### CdSe/ZnS Synthesis:

The preparation of the selenide organometallic precursor (i.e. trioctylphosphine selenide) was achieved by dissolving 0.1 mol of a selenide shot in too ml of trioctylphosphine, thereby resulting in a 1M solution of trioctylphosphine selenide. Dimethylcadmium was used as the other organometallic precursor. The CdSe precursor material (also known as quantum dots) was synthesized via the pyrolysis of dimethylcadmium and trioctylphosphine selenide in the co-ordinating trioctylphosphine oxide solvent. Precursors were injected at 3500° C. and particles/dots were grown at 2900° C. Selective size precipitation was performed with methanol to collect the particles as powders, and then they were redispersed in hexane. 5 g of trioctylphosphine oxide was heated until it reached 1900 C under a vacuum and then it was cooled to 600° C. 0.3 umol of CdSe was dispersed in hexane and transferred into the reaction vessel with the solvent being pumped off.

Hexamethyldisilathiane and diethylzinc were used as the precursors for zinc and sulphide. The average radius of the CdSe core precursors was determined from TEM, then calculating the appropriate CdSe to ZnS ratio. This was done by considering the ratio of the shell volume to that of the core and assuming a spherical core and shell and taking into account the bulk lattice parameters. Precursors were dissolved in 3 mL trioctylphosphine inside an inert atmospheric glovebox. The precursors were loaded transferred into an addition funnel, attached to a reaction flask with the CdSe cores that were dispersed in trioctylphosphine oxide. The trioctylphosphine was heated under an atmosphere of nitrogen; the precursors were then added dropwise to the reaction mixture for to minutes at a temperature of 1800° C. The mixture was then cooled to 900° C., whilst being left stirring for 3 hours; then 5 mL of butanol was added to inhibit the solidification of the trioctylphosphine oxide upon the cooling period. The nanoparticles were stored in the solution so that their surfaces remained passivated with trioctylphosphine oxide. When recovered, the powder-formed particles were precipitated with methanol and then redispersed in solvents (e.g. hexane, THF etc.).

### Chelated Gadolinium (Gd-DOTA) Silica Encapsulation:

Sodium silicate and mercaptopropyl trimethoxysilane was diluted in deionized water to a final percentage of 0.15% and 0.7%. 0.1 mL of dilute mercaptopropyl trimethoxysilane was added to a 10 mL solution of CdSe/ZnS nanoparticles and then was shaken for 20 minutes. This allows for the linking of the zinc sulphide shell with mercaptopropyl trimethoxysilane through the Zn/thiol bonds to allow for the deposition of the silica coating. 0.2 mL of the previously diluted sodium silicate solution (pH 10) was added, the solution as mixed well and was kept in a dark room at room temperature to allow for the polymerisation of the silica. After 4 hours, the solution was transferred to another vial containing 8 mL ethanol (100%) to allow the growth of a thicker silica coating due to the precipitation of the excessive silicate. The silica encapsulated nanoparticles were then precipitated out. The resulting silica encapsulated nanoparticles were added to 10 umol 1,4,7,10-Tetraazacyclododecane-1,4,7,10-tetraacetic acid (DOTA) mono-N-hydroxysuccinimide ester for 24 hours at room temperature. The gadolinium chelation to DOTA was achieved by adding two molar equivalents of the gadolinium precursor (Gd3+; GdCl3) for 24 hours at room temperature. The Gd-DOTA doped silica encapsulated nanoparticles were collected by centrifugation and washing.

### Carboxyl-Functionalization of Gd-DOTA Silica Encapsulated Nanoparticles:

40 g of Gd-DOTA silica encapsulated nanoparticles were reacted with 0.05 mmol APTES in a 1:2 deionized water-ethanol mixture (12 mL, 4 mL: 12 mL) for 24 hours in room temperature. After being aminated, to convert the terminal amine groups to carboxyl groups, the Gd-DOTA silica encapsulated nanoparticles were twice washed in ethanol and then redispersed in 20 mL of anhydrous dimethylformamide with the addition of succinic anhydride (0.06 mmol) at room temperature overnight followed by another washing with ethanol twice.

4) Conjugation with EDC

EDC (1-ethyl-3-(3-dimethylamino) propyl carbodiimide hydrochloride) is a water-soluble carbodiimide crosslinker that activates carboxyl groups for spontaneous reaction with primary amines, enabling peptide immobilisation and hapten-carrier protein conjugation. Conjugation involved covalent bonding of bispecific antibody's amine group to carboxyl groups (as described in Wen-Yen Huang et al.)

### Bispecific Antibody Conjugation to Carboxyl-Functionalized, Gd-DOTA Silica Encapsulated Nanoparticles:

25 mM carboxyl-functionalized Gd-DOTA silica encapsulated nanoparticles were coupled with the bispecific antibody (1 mg/mL). An EDC (20 mM)/sulfo-NHS (50 Mm) was prepared immediately before use. 250 μL EDC/sulfo-NHS was added to the solution of carboxyl-functionalized Gd-DOTA silica encapsulated nanoparticles. The reaction was incubated at room temperature for 10 minutes and 7 μL of 2-MEA was added to quench any excess EDC. 25 μL of the bispecific antibody solution was added to the activated carboxyl-functionalized Gd-DOTA silica encapsulated nanoparticles. The reaction was incubated room temperature for 60 minutes. Excess reactants and sulfo-NHS were removed by dialysis against Tris (pH 7.4, 50 Mm).

### 5) Aggreation

The aggregation protocol of amyloid-beta 1-42 was followed as suggested by the manufacturer (abcam http://www.abcam.com/amyloid-beta-peptide-1-42-human-ab120301.html.

Before use, and prior to opening the vial, it is recommended that the product equilibrates to room temperature for at least 1 hour. Amyloid β (1-42) human peptide should be initially dissolved at a concentration of 1 mg/ml in 100% HFIP (1,1,1,3,3,3-hexafluoro-2-propanol). This solution should be incubated at room temperature for 1 hour, with occasional vortexing at a moderate speed. Next, the solution should be sonicated for 10 minutes in a water bath sonicator. The HFIP/peptide solution should then be dried under a gentle stream of nitrogen gas. 100% DMSO should be used to re-suspend the peptide. This solution should be incubated at room temperature for 12 minutes, with occasional vortexing. The final solution should then be aliquoted into smaller volumes and stored at −80° C. For a working solution, add 500-1000 μl of D-PBS (depending on the final concentration to be used) to the peptide stock solution and incubate for 2 h at room temperature to allow for peptide aggregation. The molecular weight of the amyloid-beta species was determined by gel electrophoresis.

### 6) Surface Plasmon Resonance

Surface Plasmon resonance was performed using GE Healthcare Biacore™, the experimental setup was followed as described by the Biacore™ Assay Handbook and Biacore™ Sensor Surface Handbook:

### Nanoparticle-Probe Affinity:

Surface plasmon resonance (Biacore) was used to determine the affinity of the bispecific antibody to various targets. A 0.4M EDC/1M NHS solution was added to dextran matrix at flow rate of 10 μl/min for 7 min to activate surface. TfR solution (ligand, 50 μg/mL, PBS diluent) was added at a flow rate of 10 μl/min for 7 min. 1M ethanolamine-HCl (pH8.5) was added at flow rate of 10 μl/min for 7 min to deactivate excess reactive groups. Various concentrations of nanoparticle-probe solutions (analyte, PBS diluent) including duplicate-concentrations were used. Unmodified surface was used for reference analysis. Protocol was repeated using AB oligomers of various sizes as ligand.

### 7) Direct-Fluorescence Assay

AB monomers, oligomers, fibrils and plaques (100 pg/ml-800 pg/ml) were blocked in PBS (w/5% BSA) in 384 well plates. The probes were added and incubated at room temperature for 1 hour, then washed with PBS-T. Fluorescence was read with a plate reader at 800 nm (488 nm excitation).

### 8) Assay Kit

A standard MTT (3-(4,5-dimethylthiazol-2-yl)-2,5-diphenyltetrazolium bromide) assay kit (MTT Cell Proliferation Assay (ATCC® 30-1010K™) was used to determine the cytotoxicity of the quantum-dot probe against NIH/3T3 cells.

### 9) Immunofluorescence

Double immunofluorescence was performed was performed as described by abcam (http://www.abcam.com/ps/pdf/protocols/double %20immunofluorescence %20-simultaneous %20protocol.pdf).

### Immunofluorescence Protocol:

The coverslips were coated with polyethylineimine at room temperature for 1 hour. The coverslips were rinsed well three times with sterile water for 5 minutes each. The coverslips were allowed to dry completely and were then completely sterilized under UV light for 6 hrs. The C57Bl/Sv129 cells were grown on the glass coverslips and then rinsed briefly in phosphate-buffered saline. The cells were incubated for 30 minutes in PBST (w/1% BSA) to reduce unspecific binding. The conjugated primary antibodies (against amyloid beta 1-42 (oligomers and fibrils) and vimentin), which were stored in the dark to avoid photobleaching, were incubated with PBST overnight at 40° C. The solution was decanted and washed thrice for 5 minutes each in PBS. Cells were also incubated with 0.5 μg/ml of DAPI for 1 minute and then rinsed in PBS. Mounting medium was dropped onto the coverslip and the coverslip was sealed by applying nail polish to avoid drying. The sample was stored in the dark at −200° C. Confocal microscopy was used to characterise the results of the immunofluorescence.

### Example 1—The Nanoparticle

Referring to FIG. 1, there is shown one embodiment of a nanoparticle 2 according to the invention. The nanoparticle 2 is used to detect neurodegenerative disorders, such as Alzheimer's disease or Huntington's disease, by specifically targeting biomarkers prevalent in each disease. In addition, the nanoparticle 2 can be used to treat each disease by blocking and preventing disease development, as discussed below.

The nanoparticle 2 (also referred to herein as a “quantum dot”) consists of an inner core 4 made of cadmium selenide (CdSe), which is coated with a Zinc sulphide (ZnS) shell 6, and which is itself encapsulated with Gadolinium(Gd)-DOTA silica forming an outer shell 8. In other embodiments, the core is composed of CdTe, CdS, PbS, or PbSe etc. (instead of CdSe), the shell can be composed of CdS (instead of ZnS), and gold nanoparticles can be employed instead of Gd. A series of bispecific antibodies to or antigen-binding fragments thereof are conjugated to the Gd-DOTA silica shell 8. Each bispecific antibody 10 consists of a first Fab′ fragment 12 which is immunospecific for the transferrin receptor, i.e. it is the Fab′ fragment of IgM anti-transferrin receptor antibody.

In one embodiment, the first Fab′ fragment 12 is conjugated via its exposed sulfhydryl groups to a second Fab′ fragment 14 which is immunospecific for the oligomers and fibrils of amyloid beta protein, i.e. it is the Fab′ fragment of IgG1 anti-amyloid beta (oligomer and fibril specific) antibody; it is not immunospecific for amyloid plaques. In this first embodiment, the bispecific antibody 10 can be use in diagnosing/treating Alzheimer's disease. However, in a second embodiment, the bispecific antibody 10 can be modified for use in diagnosing/treating other neurodegenerative diseases (Parkinson's, Huntington's etc.) by switching the anti-amyloid beta (oligomer- and fibril-specific) Fab′ fragment 14 in the bispecific antibody 10 to target and thereby identify other biomarkers, e.g. alpha-synuclein oligomers and fibrils for Parkinson's disease, or Huntingtin for Huntington's disease.

The immunogen used to create the IgG1 anti-amyloid beta (oligomer and fibril specific) antibody fragment 14 was a partially aggregated recombinant peptide corresponding to human amyloid-beta (1-42) having the amino acid sequence: D-A-E-F-R-H-D-S-G-Y-E-V-H-H-Q-K-L-V-F-F-A-E-D-V-G-S-N-K-G-A-I-I-G-L-M-V-G-G-V-V-I-A (SEQ ID No:1). The bispecific antibody 2 has a low affinity to transferrin receptors due to the Fab′ fragment of IgM anti-transferrin receptor antibody, and can cross the blood-brain barrier via receptor-mediated transcytosis. The bispecific antibody 10 is specific to amyloid-beta oligomers and fibrils, whose activity leading to Alzheimer's can be detected a decade before the first symptoms are prevalent, whilst displaying low cross reactivity with amyloid-beta monomers and plaques. Fibrils are significantly larger than oligomers and are also present during the earlier stages of Alzheimer's. Therefore, it is beneficial to detect fibrils and oligomers as opposed to fibrils alone.

The nanoparticle 2 is created first by forming the inner cadmium selenide core 4, which is surrounded by the zinc sulphide shell 6. The shell 6 is then encapsulated with the carboxyl-functionalized silica shell 8 which incorporates gadolinium. The nanoparticle 2 is carboxyl-functionalized to allow for protein conjugation with the Fab′ fragments 12, 14 by reacting with their amine group using covalently bonding. The nanoparticle 2 is capable of emit light in the near infrared (NIR II) region and results in reduced autofluorescence with increase photoluminescence with the ability of non-invasive detection via functional NIR I spectroscopy. Maximum emission wavelength of quantum dots is at 845 nm with the maximum absorption wavelength being at 496 nm.

In addition, the nanoparticle 2 has MRI detection properties due to the Gd-DOTA silica shell 8.

The nanoparticle 2 with its conjugated bispecific antibody 10 renders the neurotoxic amyloid-beta oligomers insoluble (immobilised), and therefore the bound oligomers are less readily able to enter cells and their toxicity is significantly reduced.

### Example 2—Assessment of Absorption and Emission Spectra

Referring to FIG. 2, there is shown the absorption and emission spectra of the Gd-DOTA silica encapsulated CdSe/ZnS nanoparticle 2 conjugated to a bispecific antibody 10. The nanoparticles 2 display a broad absorption spectra whereas a relatively narrow emission spectra with a distinguishable peak, which is highly characteristic of quantum dots. There was also a large ‘Stokes Shift’, which would ultimately decrease fluorescence quenching and increase signal, again characteristic of quantum dots. The emission peak was at 850 nm, with the absorption peak being at 496 nm. The emission peak is in the near infrared (NIR), which can penetrate through biological tissue. Furthermore, the use of quantum dots increases the penetration depth to >2 nm as described by Hong et al.

### Example 3—Cell Viability Tests

Referring to FIG. 3 there is shown a barchart showing cell viability following exposure to the nanoparticle 2 of the invention. The nanoparticle 2 displayed very little cytotoxicity to neuronal cells (NIH/3T3), which may be credited to the silica encapsulation 8 and the ZnS shell 6 around the cadmium based core 4, with cell viability after a 48 hour incubation period being >90% in comparison to the control sample.

### Example 4—Neuronal Viability Tests

Referring to FIG. 4, there is shown a barchart showing neuronal viability following exposure to the nanoparticle 2. Oligomers and fibrils displayed a significant cytotoxicity to neuronal cells (NIH/3T3), with cell viability being at 25% and 42% respectively in comparison to the control sample. However, the bound oligomers and fibrils displayed a decreasing cytotoxicity, with cell viability with bound oligomers being 71% and bound fibrils being 83%. As such, the nanoparticle 2 displays significant therapeutic potential by decreasing the cytotoxicity of amyloid-beta fibrils and oligomers, the most neurotoxic form of amyloid-beta.

### Example 5—Affinity of the Nanoparticle to Transferrin Receptors

FIG. 5 shows the results of surface plasmon resonance employed to determine the affinity of the nanoparticle 2 to transferrin receptors. The nanoparticle 2 had a micromolar Kd (disassociation constant) value of 1.36×10⁻⁴. This high Kd value is a demonstration of a low affinity to transferrin receptors. Due to the low affinity to transferrin receptors, the nanoparticle 2 can cross the blood-brain barrier via receptor-mediated transcytosis. The anti-transferrin receptor Fab′12 in the bispecific antibody 10 was an IgM, and IgMs tend to have a naturally low affinity.

### Example 6—Fluorescence Experiments

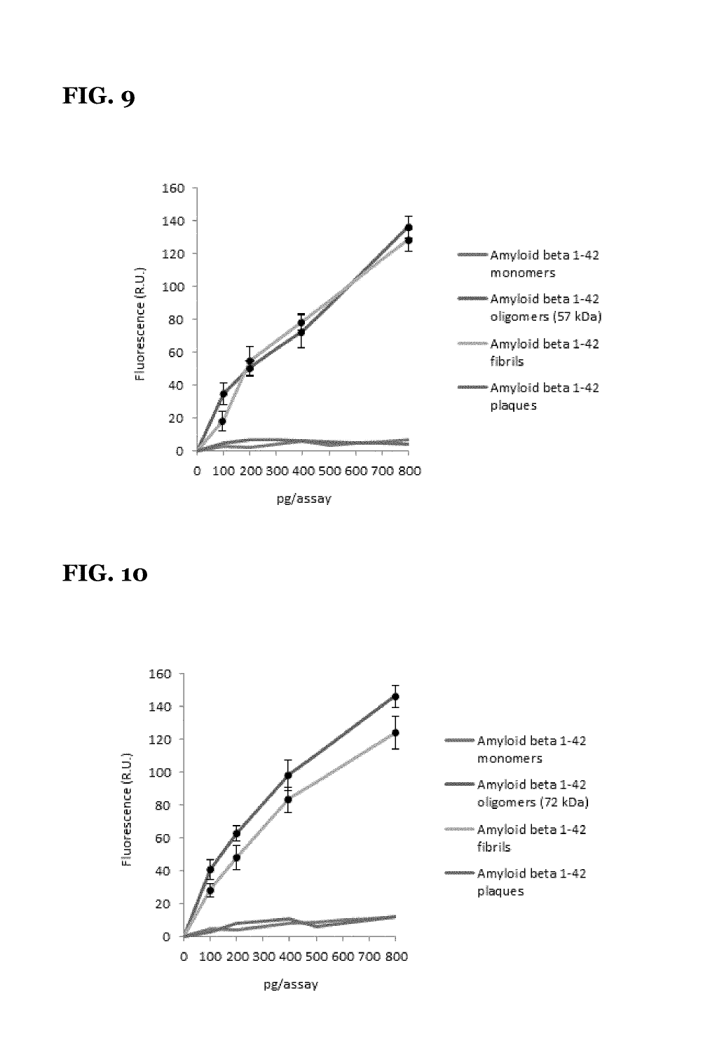

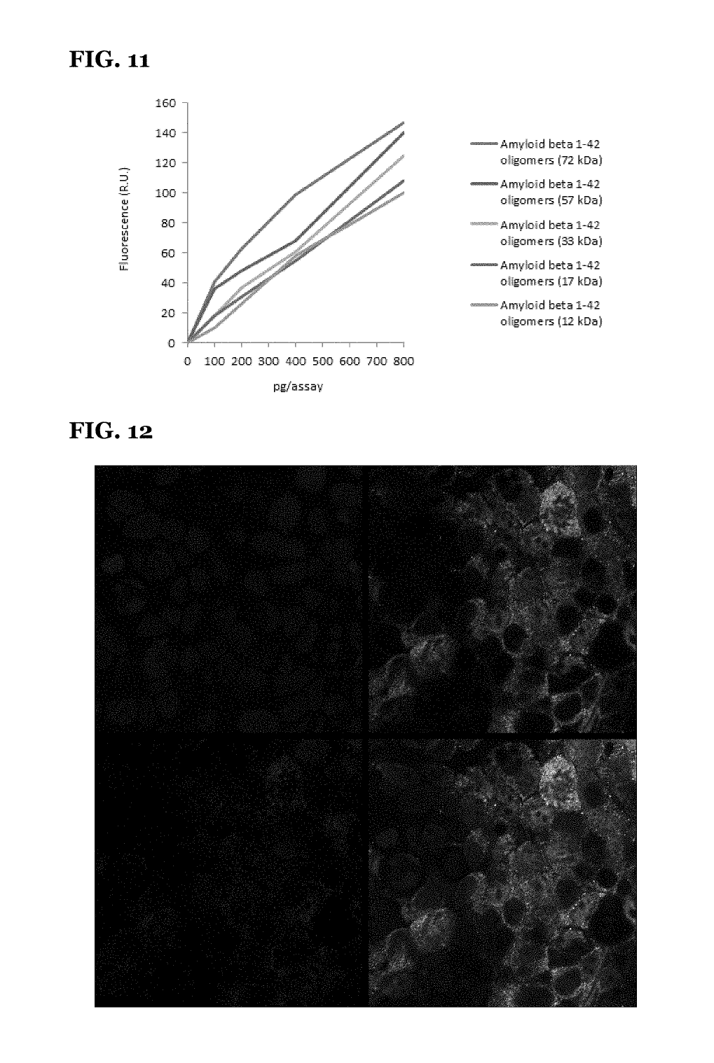

FIG. 6-11 show fluorescent data of the nanoparticle 2. As can be seen, significant fluorescence was emitted from bound fibrils and oligomers. Fluorescence emitted from fibrils stayed constant. However, as can be seen in FIG. 11, fluorescence emitted from bound oligomeric species was dependent on the ‘size’ (molecular weight) of the oligomers. FIG. 9 shows that bound oligomers of 57 kDa and above resulted in little fluorescence was emitted from monomers and fibrils, therefore demonstrating that the nanoparticle 2 had little cross-reactivity with other amyloid-beta species, thus reducing the chances of misdiagnosis.

### Example 7—Detection of Amyloid-Beta Oligomers

Referring to FIG. 12, there is shown the immunofluorescence staining of C57Bl/Sv29. FIG. 12(a) shows DAPI nuclei stain, FIG. 12(b) shows the localization of insulin, and FIG. 12(c) amyloid-beta oligomers. Success of the immunofluorescence demonstrates that the nanoparticle 2 can successfully target intracellular amyloid-beta oligomers.

FIG. 13 shows different immunofluorescence approach was taken. The nanoparticle was added to the media with the amyloid-beta oligomers and incubated for 15 minutes beforehand. In comparison to FIG. 12, there is a significant decrease in intracellular levels of amyloid-beta oligomers as fewer oligomers were able to enter the cells from the media. This demonstrates that the nanoparticle can hinder the entry of the neurotoxic protein into cells.

### Example 8—Therapeutic Potential of the Nanoparticle

FIG. 14-17 shows the results after amyloid-beta oligomers and the nanoparticle 2 were incubated together for 30+ minutes. The media containing the bound oligomers was then introduced to the neuroectodermal cells. There was extremely little intracellular amyloid beta as can be seen by the confocal images. With an incubation period of 15 minutes, as shown in FIG. 13, some amyloid-beta oligomers were still able to enter the cells from the media, however after an incubation period of 30+ minutes, very few oligomers were inside the cells. This shows that the nanoparticle 2 has therapeutic potential by binding to the neurotoxic amyloid-beta oligomers and inhibiting them from entering cells.

### DISCUSSION

The data show that the nanoparticle 2 of the invention consisting of a bispecific antibody 10 conjugated to Gd-DOTA silica outer shell 8 displayed a low cytotoxicity whilst exhibiting the ability to target intracellular amyloid-beta species. The nanoparticle 2 displayed low cross-reactivity with amyloid-beta monomers and plaques, whilst displaying a high affinity to amyloid-beta oligomers and fibrils. The nanoparticle 2 also displayed a low affinity to transferrin receptors, a required characteristic for crossing the blood brain barrier via receptor mediated transcytosis. The outer shell 8 was carboxyl functionalized to allow for direct protein conjugation of the antibody Fab′ fragments. The nanoparticles were also demonstrated to emit light in the NIR, maximally at 850 nm, due to the CdSe/ZnS composition. The Gd-DOTA silica encapsulation (i.e. the outer shell 8) significantly improves the biocompatibility of the nanoparticles 2 and drastically decreased their toxicity, and the Gd allows for potential MRI detection.

The inventor was surprised to observe the nanoparticle 2 also displayed therapeutic effects as it bound oligomers which were less readily able to enter the cells due to them being rendered insoluble, as displayed by the immunofluorescence.

An important feature of the nanoparticle 2 is the IgM anti-transferrin receptor antibody Fab′ fragment 12, as it allows the nanoparticle 2 to cross the blood-brain barrier. The IgG1 anti-amyloid beta (oligomer and fibril specific) antibody Fab′ fragment 14 used in the synthesis of the bispecific antibody can however be replaced with antibodies detecting other protein oligomers and fibrils diagnostic for other neurodegenerative diseases. For example, for Parkinson's disease, an antibody such as an anti-alpha synuclein (oligomer and fibril specific) antibody may be used to identify the biomarker characteristic of Parkinson's disease.

### REFERENCES

1. Synthesis and Characterization of Silica Coated CdSe/CdS Core/Shell Quantum Dots, (Yang Xu, Dr. Kathleen Meehan, Dr. Louis J. Guido, Dr. Guoquan Lu, Dr. Chris L. Wyatt, Dr. Nancy G. Love), December 2005, Blacksburg, Va.
2. (CdSe)ZnS Core-Shell Quantum Dots: Synthesis and Characterization of a Size Series of Highly Luminescent Nanocrystallites B. O. Dabbousi, J. Rodriguez-Viejo, F. V. Mikulec, J. R. Heine, H. Mattoussi, R. Ober, K. F. Jensen, and M. G. Bawendi
3. Gadolinium-staining reveals amyloid plaques in the brain of Alzheimer's transgenic mice. Petiet A, Santin M, Bertrand A, Wiggins C J, Petit F, Houitte D, Hantraye P, Benavides J, Debeir T, Rooney T, Dhenain M.
4. Boosting brain uptake of a therapeutic antibody by reducing its affinity for a transcytosis target. Yu Y J, Zhang Y, Kenrick M, Hoyte K, Luk W, Lu Y, Atwal J, Elliott J M, Prabhu S, Watts R J, Dennis M S. (2001 May 25)
5. Bioconjugation Protocols and Strategies Christoph M Nemeyer 2004
6. Bioconjugation Techniques Greg T Hermanson 2008
7. Alzheimer's disease: β-amyloid plaque formation in human brain Seeman P, Seeman N
8. Mechanism of amyloid plaque formation suggests an intracellular basis of Aβ pathogenicity Ralf, Friedricha, Katharina Tepper, Raik Rönickec, Malle Sooma, Martin Westermannd, Klaus Reymannc, Christoph Kaethera, and Marcus Fändricha
9. Fluorescence imaging of APP in Alzheimer's disease with quantum dot or Cy3: a comparative study. Feng L, Li S, Xiao B, Chen S, Liu R, Zhang Y. (2010 25 Mar.)
10. Structural conversion of neurotoxic amyloid-β1-42 oligomers to fibrils Mahiuddin Ahmed, Judianne Davis, Darryl Aucoin, Takeshi Sato, Shivani Ahuja, Saburo Aimoto, James I Elliott, William E Van Nostrand & Steven O Smith (2010 Mar. 5)
11. Amyloid oligomers: formation and toxicity of Ab oligomers Masafumi Sakono and Tamotsu Zak01 (6 Jan. 2010)
12. Amyloid-beta oligomerization in Alzheimer dementia versus high-pathology controls Thomas J. Esparza B S1, Hanzhi Zhao B S1, John R. Cirrito PhD1,2,3, Nigel J. Cairns PhD1,3,4, Randall J. Bateman M D1,2,3, David M. Holtzman M D1,2,3,5 and David L. Brody M D, PhD1,2,* (Dec. 7, 2012)
13. Guide to antibody labelling and detection Biomol
14. Cys diabody Quantum Dot Conjugates (ImmunoQdots) for Cancer Marker Detection Bhaswati Barat,^{†} Shannon Sirk,^{†} Katelyn McCabe,^{†} Jianqing Li,^{‡} Eric J Lepin,^{†} Roland Remenyi, Ai Leen Koh,¶ Tove Olafsen,^{†} Sanjiv S. Gambhir,§ Shimon Weiss,^{‡} and Anna M. Wut^{†}*
15. Quantum Dots for Live Cell and In Vivo Imaging Maureen A. Walling, Jennifer A. Novak and Jason R. E. Shepard * (2009)
16. Quantum Dots for Live Cell and In Vivo Imaging Maureen A. Walling, Jennifer A. Novak and Jason R. E. Shepard * (2009)

1. An amyloidogenic peptide biospecific agent comprising a nanoparticle which is visible under near infrared (NIR) and/or using Magnetic Resonance Imaging (MRI) and/or Computed Tomography (CT), and at least one antibody or antigen binding fragment thereof, which is immunospecific for a transferrin receptor and an amyloidogenic peptide.

2. A biospecific agent according to claim 1, wherein the at least one antibody or antigen binding fragment comprises an IgG anti-amyloidogenic peptide antibody or antigen binding fragment thereof.

3. A biospecific agent according to claim 1, wherein the at least one antibody or antigen binding fragment binds specifically to oligomers and fibrils of the amylodogenic peptide, but not to amyloidogenic peptide plaques or peptide monomers.

4. A biospecific agent according to claim 1, wherein the immunogen sequence used to create the at least one antibody or antigen binding fragment against the amyloidogenic peptide comprises or consists of the amino acid sequence of Aβ, or a variant or fragment thereof, preferably partially aggregated SEQ ID No:1.

5. A biospecific agent according to claim 1, wherein the immunogen sequence used to create the at least one antibody or antigen binding fragment against the amyloidogenic peptide comprises or consists of the amino acid sequence of Huntingtin, or a variant or fragment thereof, preferably partially aggregated SEQ ID No:2.

6. A biospecific agent according to claim 1, wherein the immunogen sequence used to create the at least one antibody or antigen binding fragment against the amyloidogenic peptide comprises or consists of the amino acid sequence of alpha-synuclein, or a variant or fragment thereof, preferably partially aggregated SEQ ID No:3.

7. (canceled)

8. A biospecific agent according to claim 1, wherein the at least one antibody or antigen binding fragment comprises an IgM anti-transferrin receptor antibody or antigen binding fragment thereof.

9. (canceled)

10. A biospecific agent according to claim 1, wherein the immunogen sequence used to create the at least one antibody or antigen binding fragment against the transferrin receptor comprises or consists of SEQ ID No:4 or a variant or fragment thereof.

11. A biospecific agent according to claim 1, wherein the biospecific agent comprises a plurality of antibodies or antigen binding fragments thereof with immunospecificity for a transferrin receptor, and a plurality of antibodies or antigen binding fragments thereof with immunospecificity for an amyloidogenic peptide.

12.-13. (canceled)

14. A biospecific agent according to claim 1, wherein the at least one antibody or antigen binding fragment thereof comprises an Fab′ fragment which is immunospecific for a transferrin receptor.

15. A biospecific agent according to claim 1, wherein the at least one antibody or antigen binding fragment thereof comprises an Fab′ fragment which is immunospecific for an amyloidogenic peptide, and wherein the Fab′ fragment binds specifically to oligomers and fibrils of amyloidogenic peptide, but not to amyloidogenic peptide plaques or monomers.

16. (canceled)

17. A biospecific agent according to claim 1, wherein the at least one antibody or antigen binding fragment thereof comprises a bispecific F(ab′)2 fragment which is immunospecific for an amyloidogenic peptide and a transferrin receptor, and wherein the bispecific F(ab′)2 fragment comprises a first Fab′ fragment exhibiting immunospecificity to a transferrin receptor which is conjugated to a second Fab′ fragment exhibiting immunospecificity to an amyloidogenic peptide.

18. (canceled)

19. A biospecific agent according to claim 1, wherein the nanoparticle comprises an inner core which is visible under near infrared, and wherein the core comprises cadmium or lead.

20. A biospecific agent according to claim 1, wherein the core comprises a material selected from CdSe, CdTe, CdS, PbS and PbSe.

21. (canceled)

22. A biospecific agent according to claim 1, wherein the nanoparticle comprises a cadmium or zinc shell, which surrounds the core.

23. A biospecific agent according to claim 1, wherein the shell comprises ZnS or CdS.

24. (canceled)

25. A biospecific agent according to claim 1, wherein the nanoparticle comprises a contrast material, which is visible using MRI or CT, wherein the contrast material comprises gadolinium, gold, iodine, boro-sulphate, or 1,4,7,10-tetraazacyclododecane-1,4,7,10-tetraacetic acid (DOTA).

26.-34. (canceled)

35. An NIR, MRI or CT imaging method comprising the use of the amyloidogenic peptide biospecific agent according to claim 1, optionally wherein the method is for diagnosing a neurodegenerative disorder selected from a group consisting of Alzheimer's disease; Parkinson's disease; Huntington's disease; Motor Neurone disease; Spinocerebellar type 1, type 2, and type 3; Amyotrophic Lateral Sclerosis (ALS); and Frontotemporal Dementia.

36. (canceled)

37. A kit for diagnosing a subject suffering from a neurodegenerative disorder, or a pre-disposition thereto, or for providing a prognosis of the subject's condition, the kit comprising the biospecific agent according to claim 1 configured to detect the concentration of amyloidogenic peptide present in a biological sample from a test subject, wherein presence of peptide in the sample suggests that the subject suffers from neurodegenerative disorder.

38. A method for diagnosing a subject suffering from neurodegenerative disorder, or a pre-disposition thereto, or for providing a prognosis of the subject's condition, the method comprising detecting the concentration of amyloidogenic peptide present in a biological sample obtained from a subject, wherein the detection is achieved using the biospecific agent according to claim 1, and wherein presence of antigen in the sample suggests that the subject suffers from neurodegenerative disorder.

39. (canceled)

40. A method of treating, ameliorating, or preventing a neurodegenerative disorder in a subject, the method comprising administering to a subject in need of such treatment a therapeutically effective amount of an amyloidogenic peptide biospecific agent according to claim 1.

41. The method according to claim 40, wherein the neurodegenerative disorder is selected from a group consisting of Alzheimer's disease; Parkinson's disease; Huntington's disease; Motor Neurone disease; Spinocerebellar type 1, type 2, and type 3; Amyotrophic Lateral Sclerosis (ALS); and Frontotemporal Dementia, and is preferably Alzheimer's disease.

42. (canceled)

43. A pharmaceutical composition comprising a biospecific agent according to claim 1; and optionally a pharmaceutically acceptable vehicle.

44. (canceled)


SEQUENCE LISTING (4 sequences)

SEQ ID NO: 1 - protein, 42 aa, Homo sapiens [sequence omitted]

SEQ ID NO: 2 - protein, 3144 aa, Homo sapiens
MATLEKLMKAFESLKSFQQQQQQQQQQQQQQQQQQQQQQQPPPPPPPPPPPQLPQPPPQAQPLLPQPQPPPPPPPPPPGPAVAEEPLHRPKKELSATKKDRVNHCLTICENIVAQSVRNSPEFQKLLGIAMELFLLCSDDAESDVRMVADECLNKVIKALMDSNLPRLQLELYKEIKKNGAPRSLRAALWRFAELAHLVRPQKCRPYLVNLLPCLTRTSKRPEESVQETLAAAVPKIMASFGNFANDNEIKVLLKAFIANLKSSSPTIRRTAAGSAVSICQHSRRTQYFYSWLLNVLLGLLVPVEDEHSTLLILGVLLTLRYLVPLLQQQVKDTSLKGSFGVTRKEMEVSPSAEQLVQVYELTLHHTQHQDHNVVTGALELLQQLFRTPPPELLQTLTAVGGIGQLTAAKEESGGRSRSGSIVELIAGGGSSCSPVLSRKQKGKVLLGEEEALEDDSESRSDVSSSALTASVKDEISGELAASSGVSTPGSAGHDIITEQPRSQHTLQADSVDLASCDLTSSATDGDEEDILSHSSSQVSAVPSDPAMDLNDGTQASSPISDSSQTTTEGPDSAVTPSDSSEIVLDGTDNQYLGLQIGQPQDEDEEATGILPDEASEAFRNSSMALQQAHLLKNMSHCRQPSDSSVDKFVLRDEATEPGDQENKPCRIKGDIGQSTDDDSAPLVHCVRLLSASFLLTGGKNVLVPDRDVRVSVKALALSCVGAAVALHPESFFSKLYKVPLDTTEYPEEQYVSDILNYIDHGDPQVRGATAILCGTLICSILSRSRFHVGDWMGTIRTLTGNTFSLADCIPLLRKTLKDESSVTCKLACTAVRNCVMSLCSSSYSELGLQLIIDVLTLRNSSYWLVRTELLETLAEIDFRLVSFLEAKAENLHRGAHHYTGLLKLQERVLNNVVIHLLGDEDPRVRHVAAASLIRLVPKLFYKCDQGQADPVVAVARDQSSVYLKLLMHETQPPSHFSVSTITRIYRGYNLLPSITDVTMENNLSRVIAAVSHELITSTTRALTFGCCEALCLLSTAFPVCIWSLGWHCGVPPLSASDESRKSCTVGMATMILTLLSSAWFPLDLSAHQDALILAGNLLAASAPKSLRSSWASEEEANPAATKQEEVWPALGDRALVPMVEQLFSHLLKVINICAHVLDDVAPGPAIKAALPSLTNPPSLSPIRRKGKEKEPGEQASVPLSPKKGSEASAASRQSDTSGPVTTSKSSSLGSFYHLPSYLKLHDVLKATHANYKVTLDLQNSTEKFGGFLRSALDVLSQILELATLQDIGKCVEEILGYLKSCFSREPMMATVCVQQLLKTLFGTNLASQFDGLSSNPSKSQGRAQRLGSSSVRPGLYHYCFMAPYTHFTQALADASLRNMVQAEQENDTSGWFDVLQKVSTQLKTNLTSVTKNRADKNAIHNHIRLFEPLVIKALKQYTTTTCVQLQKQVLDLLAQLVQLRVNYCLLDSDQVFIGFVLKQFEYIEVGQFRESEAIIPNIFFFLVLLSYERYHSKQIIGIPKIIQLCDGIMASGRKAVTHAIPALQPIVHDLFVLRGTNKADAGKELETQKEVVVSMLLRLIQYHQVLEMFILVLQQCHKENEDKWKRLSRQIADIILPMLAKQQMHIDSHEALGVLNTLFEILAPSSLRPVDMLLRSMFVTPNTMASVSTVQLWISGILAILRVLISQSTEDIVLSRIQELSFSPYLISCTVINRLRDGDSTSTLEEHSEGKQIKNLPEETFSRFLLQLVGILLEDIVTKQLKVEMSEQQHTFYCQELGTLLMCLIHIFKSGMFRRITAAATRLFRSDGCGGSFYTLDSLNLRARSMITTHPALVLLWCQILLLVNHTDYRWWAEVQQTPKRHSLSSTKLLSPQMSGEEEDSDLAAKLGMCNREIVRRGALILFCDYVCQNLHDSEHLTWLIVNHIQDLISLSHEPPVQDFISAVHRNSAASGLFIQAIQSRCENLSTPTMLKKTLQCLEGIHLSQSGAVLTLYVDRLLCTPFRVLARMVDILACRRVEMLLAANLQSSMAQLPMEELNRIQEYLQSSGLAQRHQRLYSLLDRFRLSTMQDSLSPSPPVSSHPLDGDGHVSLETVSPDKDWYVHLVKSQCWTRSDSALLEGAELVNRIPAEDMNAFMMNSEFNLSLLAPCLSLGMSEISGGQKSALFEAAREVTLARVSGTVQQLPAVHHVFQPELPAEPAAYWSKLNDLFGDAALYQSLPTLARALAQYLVVVSKLPSHLHLPPEKEKDIVKFVVATLEALSWHLIHEQIPLSLDLQAGLDCCCLALQLPGLWSVVSSTEFVTHACSLIYCVHFILEAVAVQPGEQLLSPERRTNTPKAISEEEEEVDPNTQNPKYITAACEMVAEMVESLQSVLALGHKRNSGVPAFLTPLLRNIIISLARLPLVNSYTRVPPLVWKLGWSPKPGGDFGTAFPEIPVEFLQEKEVFKEFIYRINTLGWTSRTQFEETWATLLGVLVTQPLVMEQEESPPEEDTERTQINVLAVQAITSLVLSAMTVPVAGNPAVSCLEQQPRNKPLKALDTRFGRKLSIIRGIVEQEIQAMVSKRENIATHHLYQAWDPVPSLSPATTGALISHEKLLLQINPERELGSMSYKLGQVSIHSVWLGNSITPLREEEWDEEEEEEADAPAPSSPPTSPVNSRKHRAGVDIHSCSQFLLELYSRWILPSSSARRTPAILISEVVRSLLVVSDLFTERNQFELMYVTLTELRRVHPSEDEILAQYLVPATCKAAAVLGMDKAVAEPVSRLLESTLRSSHLPSRVGALHGVLYVLECDLLDDTAKQLIPVISDYLLSNLKGIAHCVNIHSQQHVLVMCATAFYLIENYPLDVGPEFSASIIQMCGVMLSGSEESTPSIIYHCALRGLERLLLSEQLSRLDAESLVKLSVDRVNVHSPHRAMAALGLMLTCMYTGKEKVSPGRTSDPNPAAPDSESVIVAMERVSVLFDRIRKGFPCEARVVARILPQFLDDFFPPQDIMNKVIGEFLSNQQPYPQFMATVVYKVFQTLHSTGQSSMVRDWVMLSLSNFTQRAPVAMATWSLSCFFVSASTSPWVAAILPHVISRMGKLEQVDVNLFCLVATDFYRHQIEEELDRRAFQSVLEVVAAPGSPYHRLLTCLRNVHKVTTC

SEQ ID NO: 3 - protein, 140 aa, Homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 4 - protein, 760 aa, Homo sapiens
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF